(19) United States
(12) Patent Application Publication
(10) Pub. No.: US 2025/0186751 A1
(43) Pub. Date: Jun. 12, 2025

(54) BREATH-POWERED NASAL DEVICES FOR TREATMENT OF TRAUMATIC BRAIN INJURY (TBI), INCLUDING CONCUSSION, AND METHODS AND METHODS

(71) Applicant: Odyssey Health, Inc., Las Vegas, NV (US)

(72) Inventors: Jacob VanLandingham, Tallahassee, FL (US); Michael Lewandowski, Odessa, FL (US); Kelly M. Stowell, Costa Mesa, CA (US); Jonathan Lucas, Long Beach, CA (US); Travis Cochran, Costa Mesa, CA (US)

(21) Appl. No.: 17/803,705

(22) Filed: Oct. 19, 2022

Related U.S. Application Data
(60) Provisional application No. 63/257,117, filed on Oct. 19, 2021.

(51) Int. Cl. A61M 31/00

(52) U.S. Cl. CPC A61M 31/00; A61M 2202/0007; A61M 2205/0216; A61M 2205/3331; A61M 2206/20; A61M 2210/0618; A61M 2210/0625

(57) ABSTRACT

The present invention is directed to a single-directional insufflator or breath-powered nasal device that provides unique dual airflow for propelling a drug substance into a nasal cavity, preferably deep into the superior nasal cavity and into the olfactory region and the trigeminal nerve mucosa, for rapid diffusion into the brain for the treatment of nasal and/or central nervous system (“CNS”) injury, disease or disorder, especially brain injury, such as traumatic brain injury (“TBI”), including concussion, and methods regarding nasal treatment therewith.

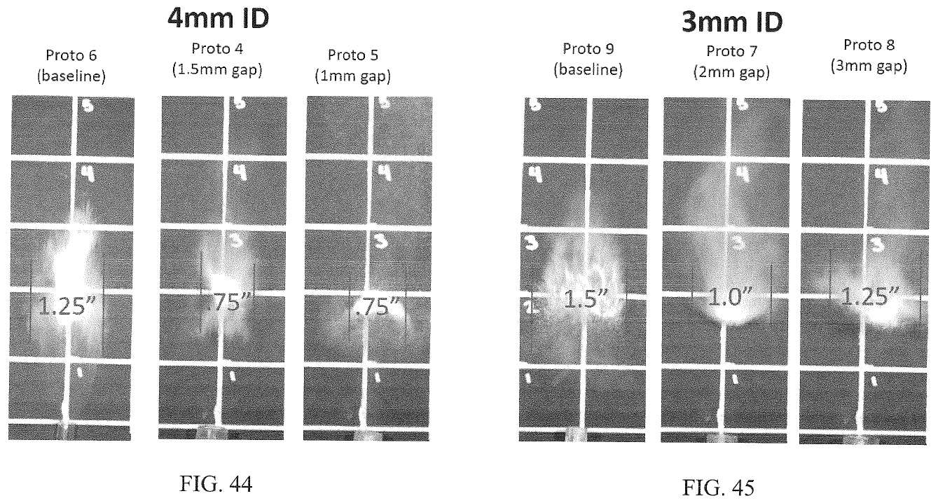

Breath-powered nasal devices for delivering a somewhat tight or confined, concentrated plume of drug substance deep into the nasal cavity to targeted sites, bypassing the blood brain barrier, namely the olfactory nerve to provide concentrated drug substance for direct absorption into the brain are disclosed. The confined plume of drug substance propelled from the nasal device by a patient's breath is narrow in the vertical resembling the diameter of the inner chamber in which the drug substance is stored. By forming this narrow or confined plume, a greater concentration of the drug substance can be deposited at the deeper nasally targeted sites. The breath-powered nasal devices of the present invention accomplish this unique advantage by the use of a novel dual elongated double-walled tube chamber comprised of an outer hollow chamber and the inner chamber in which the inner chamber is stabilized in the outer chamber by preferably two, three or more ribs. More specifically, the breath-powered nasal device uses the patient's own breath to blow air simultaneously at the same force rate through both chambers. As the blown air exits both chambers, the propelled air from the outer tube chamber surrounds the drug substance propelled from the inner tube chamber within the blown air forcing the drug substance to exit in a tight or confined, concentrated plume shape in the vertical, so that a higher concentration of drug substance reaches the targeted nasal sites, as compared to a conical fanned-out plume shape of drug substance exiting a single hollow chamber, as illustrated in FIGS. 44-45. This unique feature allows diseases or injuries of the brain to be treated more effectively with a drug substance without or with minimized systemic or Blood Brain Barrier (“BBB”) issue.

### RELATED U.S. PROVISIONAL APPLICATION

This utility application for U.S. patent claims priority from U.S. provisional application, Ser. No. 63/257,117, which was filed on Oct. 19, 2021, of which the foregoing U.S. provisional application is incorporated herein by reference in its entirety as if fully set forth herein.

### FIELD OF THE INVENTION

The present invention is directed to a single-directional insufflator or breath-powered nasal device that provides unique dual airflow for propelling a drug substance into a nasal cavity, preferably deep into the superior nasal cavity and into the olfactory region and the trigeminal nerve mucosa, for rapid diffusion into the brain for the treatment of nasal and/or central nervous system (“CNS”) injury, disease or disorder, especially brain injury, such as traumatic brain injury (“TBI”), including concussion, and methods regarding nasal treatment therewith.

### BACKGROUND

The noninvasive nature of intranasal drug administration makes it an attractive option for local and systemic delivery of therapeutic agents. See FDA. Content current as of: Apr. 28, 2021. How computational analysis of a 3D mucociliary clearance model can help predict drug uptake and lead to more generic nasal drug products. https://www.fda.gov/drugs/regulatory-science-research-and-education/how-computational-analysis-3d-mucociliary-clearance-model-can-help-predict-drug-uptake-and-lead-more (“FDA, Apr. 28, 2021”). The nasal mucosa, unlike other mucosae, is easily accessible. FDA, Apr. 28, 2021. Intranasal application circumvents the issues of gastrointestinal degradation and hepatic first-pass metabolism of drugs. FDA, Apr. 28, 2021. See also Bitter C, Suter-Zimmermann K, Surber C.: Nasal drug delivery in humans. Karger Publishers, Topical Applications and the Mucosa. pages 20-35 (2011).

Drugs intended to hit a target site within the nasal cavity often get trapped in the highly viscous gel layer, which can reduce drug efficacy. FDA, Apr. 28, 2021. Notwithstanding the trapping, soluble drugs dissolve in the mucus layer, diffuse across the gel and sol layers, and are eventually absorbed by the richly vascularized nasal epithelium. FDA, Apr. 28, 2021. It is believed that this process within the nasal housing enables a drug to enter the systemic regions through the blood stream without losing efficacy. FDA, Apr. 28, 2021.

For most nasal delivery purposes, a broad distribution of the drug on the mucosal surfaces appears acceptable for drugs that are intended for local action or systemic absorption. See, e.g., Vidgren M T, Kublik H.: Nasal delivery systems and their effect on deposition and absorption. Adv Drug Deliv Rev. 29:157-77 (1998). See also Djupesland, P. G.: Nasal drug delivery devices: characteristics and performance in a clinical perspective—a review. Drug Deliv. and Transl. Res. 3:42-62 (2013). In chronic sinusitis and nasal polyposis, however, strategic targeted delivery to the middle and superior meatuses at which the sinus openings are located and the polyps originate, is beneficial. See Laube B.: Devices for aerosol delivery to treat sinusitis. J Aerosol Med. 20 (Suppl): 5-18. 43 (2007); and Aggrawal R, Cardozo A, Homer J J.: The assessment of topical nasal drug distribution. Clin Otolaryngol. 29:201-5 (2004). See also Djupesland, P. G.: Nasal drug delivery devices: characteristics and performance in a clinical perspective—a review. Drug Deliv. and Transl. Res. 3:42-62 (2013).

The above notwithstanding, there is an interest within industry for developing products that target blood-brain barrier (BBB) for rapid delivery to the central nervous system, including to the brain. Pardeshi C V, Belgamwar V S.: Direct nose to brain drug delivery via integrated nerve pathways bypassing the blood-brain barrier: an excellent platform for brain targeting. Expert opinion on drug delivery 10 (7): 957-72. (2013). See also FDA, Apr. 28, 2021. Unfortunately, the most recent FDA guidance for nasal devices appears to essentially address mechanical liquid spray pumps and pressurized metered-dose inhalers for local action. FDA 2003. US FDA draft guidance for industry. Bioavailability and bioequivalence studies for nasal aerosols and nasal sprays. Bethesda. http://www.fda.gov/cder/guidance/index.htm. April 2003. See also Djupesland, P. G.: Nasal drug delivery devices: characteristics and performance in a clinical perspective—a review. Drug Deliv. and Transl. Res. 3:42-62 (2013) (“FDA 2003 Guidance”). See also Djupesland, P. G.: Nasal drug delivery devices: characteristics and performance in a clinical perspective—a review. Drug Deliv. and Transl. Res. 3:42-62 (2013). The FDA guidance apparently offers no guidance for breath-powered or breath-propelled nasal devices for nasal use for delivering drug substances into the brain for treatment of brain injury or disease. FDA 2003 Guidance. See also Djupesland, P. G.: Nasal drug delivery devices: characteristics and performance in a clinical perspective—a review. Drug Deliv. and Transl. Res. 3:42-62 (2013).

Unlike broad nasal drug substance distribution within the nasal cavity for local action or systemic absorption, drug substances intended for “nose-to-brain” (NTB) delivery present unique challenge. NTB drug delivery requires a more targeted delivery to the upper nasal parts located deep inside the nasal cavity of the nose housing for deposition into the olfactory region and onto the olfactory nerves which is essential for NTB drug delivery and effective brain treatment. Djupesland, P. G.: Nasal drug delivery devices: characteristics and performance in a clinical perspective—a review. Drug Deliv. and Transl. Res. 3:42-62 (2013).

Recent animal data suggest, however, that some degree of drug substance transport may occur along and across the branches of the first and second divisions of the trigeminal nerve innervating most if not all of the mucosa at and well beyond the nasal valve. Johnson H. J. et. al.: Trigeminal pathways deliver a low molecular weight drug from the nose to the brain and orofacial structures. Mol Pharm. (3): 884-93 (2010). See also Djupesland, P. G.: Nasal drug delivery devices: characteristics and performance in a clinical perspective—a review. Drug Deliv. and Transl. Res. 3:42-62 (2013). Johnson H. J. et al. therefore suggests that a combination of targeted delivery to the olfactory region and a broad distribution to the mucosa innervated by the trigeminal nerve may be optimal for NTB delivery and effective brain treatment by a drug substance. Djupesland, P. G.: Nasal drug delivery devices: characteristics and performance in a clinical perspective—a review. Drug Deliv. and Transl. Res. 3:42-62 (2013).

The central nervous system (CNS) includes the brain, the brain stem, and the spinal cord. The CNS is isolated from the external world by several membranes that both cushion and protect the brain, the brain stem, and the spinal cord. For example, the membranes that form the blood-brain barrier (BBB) protect the brain from certain contents of the blood. The blood-cerebrospinal fluid barrier (BCSFB) protects other portions of the CNS from many chemicals and microbes. Thus, a two barrier system in brain forms the BBB, i.e., the brain microvascular endothelium and the choroid plexus epithelium (BCSFB). In addition to the BBB, there is an arachnoid epithelium barrier around the brain that creates the sub-arachnoid space.

Drug transport from blood to cerebrospinal fluid (CSF) is regulated by the choroid plexus, or BCSFB. Drug transport from blood to interstitial fluid (ISF) is regulated by the brain microvascular endothelium, or BBB. See W. M. Pardridge: Drug transport in brain via the cerebrospinal fluid. Fluids Barriers CNS. 8:7 (2011).

There is no lymphatic system in the human brain. Nevertheless, more than 500 mls of cerebrospinal fluid is produced by the human brain every day. The CSF is secreted at the choroid plexus, or the BCSFB. The CSF fills the cranial and spinal sub-arachnoid space as well as the cavities of the four ventricles. See W. M. Pardridge: Drug transport in brain via the cerebrospinal fluid. Fluids Barriers CNS. 8:7 (2011). The ventricles of the brain are a communicating network of cavities occupied with the CSF and are located within the brain parenchyma. The ventricular system comprises two lateral ventricles, a third ventricle, a cerebral aqueduct, and a fourth ventricle. The choroid plexuses are located in the ventricles that secrete the CSF, which then fills the ventricles and subarachnoid space, following a cycle of constant production and reabsorption. See https://emedicine.medscape.com/article/1923254-overview#:˜:text-Overview-,Overview,(see%20the%20images%20below).

The CSF flows over the surfaces of the brain and spinal cord. The CSF is rapidly absorbed into the general circulation. As discussed above, the choroid plexus forms the blood-cerebrospinal fluid barrier, and this barrier is functionally distinct from the brain microvascular endothelium, which forms the blood-brain barrier. Virtually all non-cellular substances in blood distribute into cerebrospinal fluid, and drug entry into cerebrospinal fluid is not an index of drug transport across the blood-brain barrier. Drug injected into the cerebrospinal fluid rapidly moves into the blood via bulk flow, but penetrates into brain tissue poorly owing to the limitations of diffusion. See W. M. Pardridge: Drug transport in brain via the cerebrospinal fluid. Fluids Barriers CNS. 8:7 (2011).

Conventional methods for delivering compounds to the CNS are generally invasive. For example, certain compounds, like epidural steroid injections (ESI), facet joint injections, hardware injections, sacroiliac (SI) joint injections and differential lower extremity injections, are injected directly through the protective membrane into the CNS. However, spinal injections are not trivial and are generally impractical for most compounds.

Another example for delivering a compound to the CNS is a pump implanted in the skull, such as an intracerebroventricular (ICV) pump, can deliver a variety of compounds to the brain. However, implanting such a pump requires brain surgery, which can entail a variety of serious complications. Nevertheless, ICV, or intraventricular, devices have been used in the treatment of a broad range of pediatric and adult central nervous system (CNS) disorders. Because of limited permeability of the blood brain barrier, CNS diseases generally require direct administration of drugs into the brain to achieve meaningful therapeutic effect. It is generally accepted that the clinical use and complications of ICV drug delivery result in device-associated complication rates as high as 33% for non-infectious complications and 27% for infectious complications. These undesirable safety outcomes may be due a lack of agreement amongst practioners on best practices of device use. See Slave I. et al.: Best practices for the use of ICV drug delivery devices. Molecular Genetics and Metabolism. 124 (3): 184-188 (2018).

Much of current brain research is focused on the enhancement of the drug being delivered to the brain by various formulations. The traditional approaches to improve uptake of compounds to the brain include (1) mucoadhesive formulations; 2) penetration enhancers; 3) liposomes; 4) vasoconstrictors; and 5) nanoparticles. Examples of various compounds with have enhanced formulations include various cytokines, for example, tumor necrosis factors, interleukins, and interferons discussed in U.S. Pat. No. 6,991,785 and growth and differentiation factor-5 (GDF-5) and related proteins discussed in U.S. Publication No. 20100074959.

Targeting of drugs to the central nervous system (CNS) is a challenging task. A great number of drugs, including biotechnology products, are candidates for treatment of CNS diseases, but drug delivery through the BBB is a problem for brain targeting. A limitation in the treatment of brain tumors is that less than 1% of most therapeutic agents administered systemically are able to cross the BBB. The transport of small molecules across the BBB is the exception rather than the rule, and 98% of all small molecules do not cross the BBB. See, e.g., Pardride, W. M: The blood-brain barrier: bottleneck in brain drug development. NeuroRx. 2 (1): 3-14 (January 2005), in which Pardride concludes that approximately 100% of large-molecule drugs or genes do not cross the BBB. According to Pardride, the BBB allows small (about less than 500 Da), lipophilic molecules from the bloodstream to enter the CNS and that many larger therapeutic agents are prevented from reaching the brain for treating CNS disorders such as but not limited to Parkinson's disease, Alzheimer's disease, depression, stroke, and epilepsy. Pardride, W. M: The blood-brain barrier: bottleneck in brain drug development. NeuroRx. 2 (1): 3-14 (January 2005). See also Chapman, C. D. et. al.: Intranasal Treatment of the Central Nervous System Dysfunction in Humans. Pharm. Res. 30:2475-2484 (2013).

Disorders including autism, lysosomal storage disorders, fragile X syndrome, ataxis, and blindness, are serious disorders where there is little effective treatment. In many of these cases, the gene underlying the disease is known, but BBB delivery is the rate-limiting problem in gene therapy or enzyme replacement therapy, and no therapeutics have been developed. Drug delivery of therapeutic compounds, for example proteins, faces several challenges because of their instability, high enzymatic metabolism, low gastrointestinal absorption, rapid renal elimination, and potential immunogenicity. Pardride, W. M: The blood-brain barrier: bottleneck in brain drug development. NeuroRx. 2 (1): 3-14 (January 2005).

Pardridge believes that “ . . . despite the importance of the BBB to the neurotherapeutics mission, the BBB receives insufficient attention in either academic neuroscience or industry programs and that combination of so little effort in developing solutions to the BBB problem, and the minimal BBB transport of the majority of all potential CNS drugs, leads predictably to the present situation in neurotherapeutics, which is that there are few effective treatments for the majority of CNS disorders.” Pardride, W. M: The blood-brain barrier: bottleneck in brain drug development. NeuroRx. 2 (1): 3-14 (January 2005). See also FDA 2003 Guidance.

According to Chapman, C. D. et. al., intranasal delivery is emerging as a noninvasive option for delivering drugs to the CNS with minimal peripheral exposure. Chapman, C. D. et. al.: Intranasal Treatment of the Central Nervous System Dysfunction in Humans. Pharm. Res. 30:2475-2484 (2013). See also FDA, Apr. 28, 2021; Trevino, J. T. et al.: Non-Invasive Strategies for Nose-to-Brain Drug Delivery. J Clin Trials. 10 (7): 439. (2020); and Dhakar, R. C. et. al.: Nasal Drug Delivery: Success Through Integrated Device Development. J. Drug Deliv. Ther. 1 (1): 2-7 (2011). Chapman, C. D. et. al. suggest that intranasal delivery facilitates the delivery of large and/or charged therapeutics, which fail to effectively cross the blood-brain barrier (BBB). Thus, Chapman, C. D. et. al. concludes that for a variety of growth factors, hormones, neuropeptides and therapeutics including insulin, oxytocin, orexin, and even stem cells, intranasal delivery is materializing as an efficient method of administration, and represents a promising therapeutic strategy for the treatment of diseases with CNS involvement, such as obesity, Alzheimer's disease, Parkinson's disease, Huntington's disease, depression, anxiety, autism spectrum disorders, seizures, drug addiction, eating disorders, and stroke. Chapman, C. D. et. al.: Intranasal Treatment of the Central Nervous System Dysfunction in Humans. Pharm. Res. 30:2475-2484 (2013).

However, intranasal administration has traditionally focused on the distribution of drug solutions as a mist for topical delivery to the nasal epithelium. Because of the nasal cavity's easily accessed vascular bed, nasal administration of medications has focused the delivery of medications either locally to the nasal cavity or directly to the blood stream.

There is a definite need for nasal insufflators that can deliver drug substances to the upper nasal cavity, thereby avoiding the BBB problems, for direct nose-to-brain or NTB delivery for effective treatment of brain injury and disorders. Certain existing nasal drug delivery devices do not adequately propel the drug from the device or concentrate the drug for effective targeting and treatment. Inconsistent propulsion of a steady flow of drug concentration due is also far from optimal. Still further, the plume generated by such existing devices is far too wide and scattered for effective NTB delivery. Certain existing nasal drug devices rely on circumferential velocity to propel medicaments to the olfactory epithelium. Traditional circumferential devices result in a lower percentage of drug substance being deposited on the olfactory epithelium. A circumferential component in the aerosol plume tends to result in a wider spray plume with a portion of the aerosol particles targeted to the sides of the nasal cavity in the lower part of the nasal cavity.

Thus, there is a definite clinical unmet need for improved nasal insufflators and mechanisms that effectively target and deliver concentrated depositions of desired drug substances within the brain, brain stem, and/or spinal cord and improve clinical performance and outcomes in the treatment of CNS and brain injury and disorders, such as TBI, especially concussions.

### SUMMARY OF THE INVENTION

The present invention overcomes the above-mentioned disadvantages and drawbacks of the present state of nasal insufflator art through the discovery of a novel single-directional insufflator or breath-powered nasal device and methods that provide unique dual airflow for propelling a drug substance into a nasal cavity, preferably deep into the superior nasal cavity for effective targeted delivery to the olfactory region and distribution into the mucosa innervated by the trigeminal nerve in high drug substance concentration for rapid diffusion into the brain for the treatment of nasal and/or central nervous system (“CNS”) injury, disease or disorder, especially brain injury, including traumatic brain injury (“TBI”), especially concussions.

The novel single-directional insufflator or breath-powered nasal devices and delivery methods of the present invention utilize both a novel dual wall drug dispensing system, i.e., a double walled-tube configuration, and the natural nasal anatomy and features to improve nasal delivery of drug substances in the form of fine particles, liquids, suspensions, gels and the like without pulmonary or gastric deposition or first-pass liver metabolism of the drug substance, and thus to improve nasal treatment efficacy. The novel single-directional insufflator or breath-powered nasal devices are preferably disposable, single-use devices for mono dose delivery of a single drug substance or for mono dose delivery of two or more drugs substances. The novel single-directional insufflator or breath-powered nasal devices of the present invention are hygienic and provide unique effective and accurate dosing of a drug substance for effective nasal treatment of local, systemic and CNS injury, disease or disorders.

Use of a novel single-directional insufflator or breath-powered nasal device of the present invention is intuitively simple and takes advantage of the natural nasal reflex for blowing a dry-powder or liquid dose deep into the nasal cavity for effective targeted delivery to the olfactory region and distribution into the mucosa innervated by the trigeminal nerve in high drug substance concentration for rapid diffusion into the brain. To illustrate, a subject using a device of the present invention snugly fits the nose guard piece on one end of the device into one nostril and then inserts or positions the mouth piece on the other end of the device into the mouth between the lips. When the subject exhales blown air through the inserted or positioned mouth piece against resistance generated from the device, the nasal reflex that automatically closes the soft palate is activated thereby closing the subject's oral and nasal airway passages. As a result, the blown drug substance loaded into the device is propelled from the nose guard, within the exiting unique dual airflow created by the novel dual wall drug dispensing system of the device, and is delivered and trapped deep within the nasal cavity for delivering the trapped drug substance to the superior nasal membranes within the olfactory region and the trigeminal nerve mucosa for diffusion directly into the brain within minutes of device use. Once the subject's blow for dose delivery to the nasal cavity is complete, the subject's nasal reflex naturally subsides and the subject's oral and nasal airway passages re-open.

While any effective blown airflow rate is contemplated by the present invention, a blown airflow rate of about 40 Liters per Minute at a pressure of at least about 40 cm H₂O pressure or greater appears to be more than suitable to effectively operate a novel single-directional insufflator or breath-powered nasal device of the present invention.

It is therefore among objectives of this invention to provide a novel single-directional insufflator or breath-powered nasal device intended for treatment of local, systemic and CNS injury, disease and disorders, especially TBI and more especially concussion, through the use of a novel and unique combination of dual airflow to propel highly concentrated drug substance deep into a nasal cavity for deposition into and distribution throughout the olfactory region and distribution into the mucosa innervated by the trigeminal nerve and for diffusion directly into the brain thereby uniquely avoiding systemic and BBB issues. The novel single-directional insufflator or breath-powered nasal device may, if so elected, also deposit a drug substance within the nasal cavity for local and systemic application of the same or different drug substances. These objectives are accomplished by the novel and unique dual wall drug dispensing system configured within the novel single-directional insufflator or breath-powered nasal device of the present invention.

Generally speaking, the single-directional insufflator or breath-powered nasal devices of the present invention comprises a one-way valve mouth piece at one end and a nose guard at the other end that are fluidly connected via a corrugated tubing for delivering a loaded drug substance deep within the nasal cavity through oral pressurization by blowing through the one-way valve mouth piece and through the novel dual-wall drug-substance technology to cause the drug substance to exit the nose guard in a vertical, somewhat tight and narrow or confined, concentrated plume for delivering a high drug substance concentration to the targeted sites deep within the nasal cavity and distributing throughout the olfactory region and into the mucosa innervated by the trigeminal nerve and for diffusing therefrom into the brain. Delivery of the drug substance, in for example, dry powder, liquid, suspension or gel form and the like, occurs when the nose guard is inserted snugly into one nostril of a person and the person blows into the one-way valve mouth piece, which breath closes the person's velum (soft palate) and causes the pressurized airflow to travel through the one-way valve mouth piece and then through the novel dual wall drug dispensing system of the present invention, which upon nose guard exit, uniquely carries the drug substance in the vertical, somewhat tight or confined and narrow, concentrated plume shape in high drug concentration deep into the superior nasal cavity for effective distribution in the olfactory region and the mucosa innervated by the trigeminal nerve and for direct diffusion therefrom into the brain.

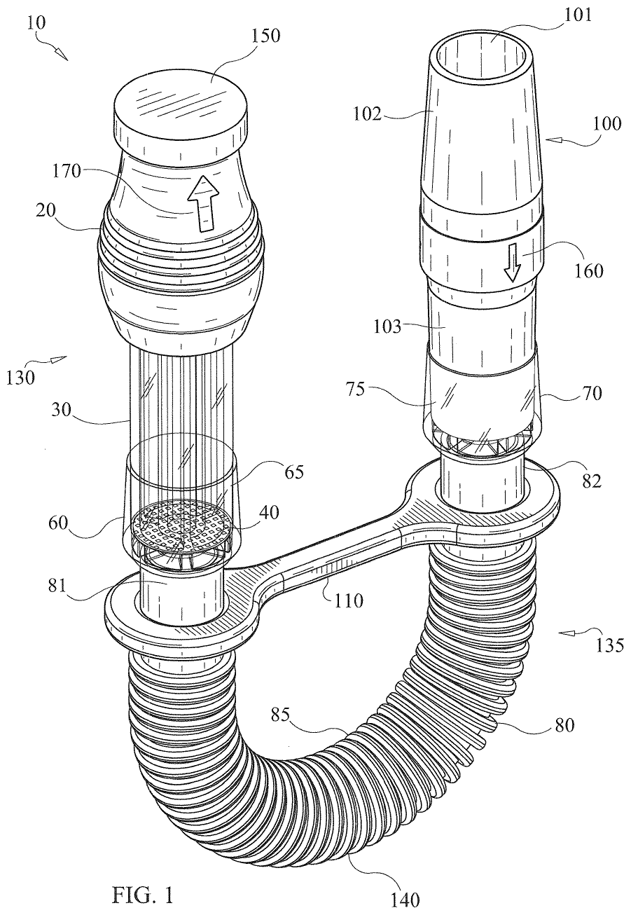

More specifically, and as shown in FIGS. 1-2 and 9-10, single-directional insufflator or breath-powered nasal device 10 of the present invention comprises nose guard 20, dual wall drug dispensing system 30, mesh disc 40 on which drug 200 (not shown) is positioned, first tube connector 60, corrugated tubing 80, second tube connector 70, ridge connector 110, one-way valve mouth piece 100 housing a one-way valve 50 (not shown) in bottom section 103 that directs airflow in one direction and prevents reverse airflow direction, so that, first air-flow directional indicator band 160 and second air-flow directional indicator band 170, when single-directional insufflator or breath-powered nasal device 10 is in use and seal cap 150, i.e., top cover, is removed and user blows air through the one-way vale mouth piece 100, drug 200 (not shown) positioned on mesh disc 40 is propelled out of nose guard 20.

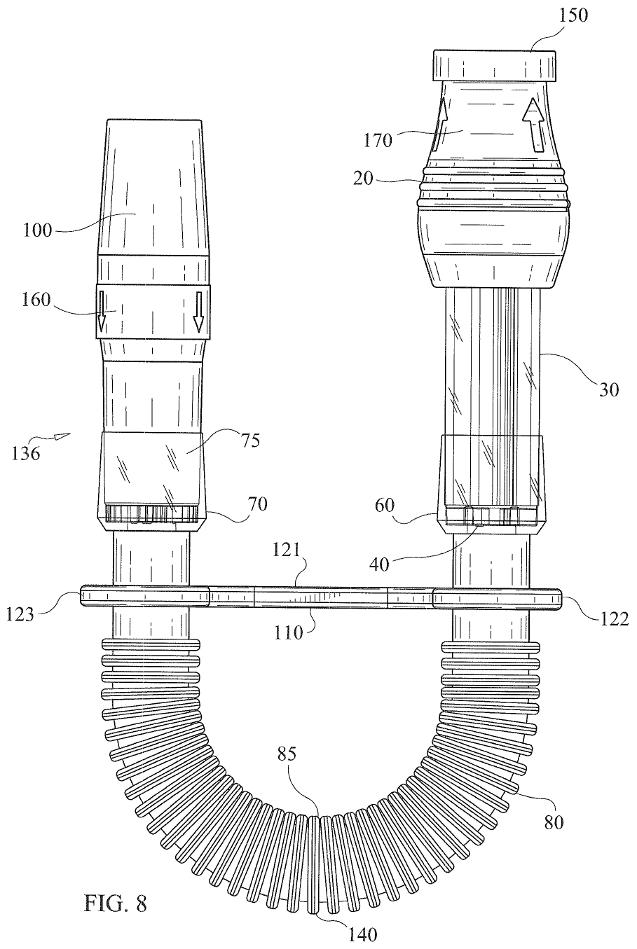

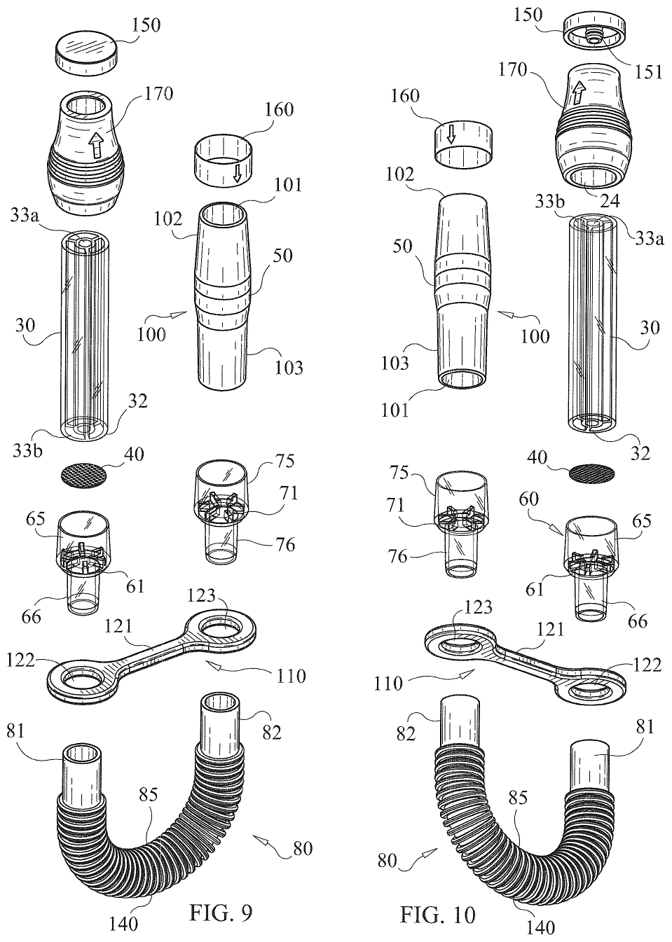

As further shown in FIGS. 9-10, the top end portion 31 of the dual wall drug dispensing system 30 is press-fit through bottom 26 of through-hole 24 of nose guard 20 and positioned snugly against nose guard seat 22 located near top 25 of through-hole 24 of nose guard 20. The bottom end portion 32 of the dual wall drug dispensing system 30 is press-fit into first top hollow collar 65 of first tube connector 60 to form a tight seal there between. Mesh disc 40 is inserted into and sits on top of first seat 61 located at the bottom of first top hollow collar 65 of first tube connector 60. First bottom hollow neck 66 of first tube connector 60 is press-fit into the first hollow cuff 81 of corrugated tubing 80 to form a tight seal there between. Second bottom hollow neck 76 of second tube connector 70 is press-fit into the second hollow cuff 82 of corrugated tubing 80 to form a tight seal there between. Bottom section 102 of one-way valve mouth piece 100 is press-fit into second top hollow collar 75 of second tube connector 70 until it is resting on second seat 71 of second tube connector 70 to form a tight seal there between.

Thus, and as further shown in FIGS. 9-10, one-way valve 50 (not shown) housed in bottom section 103 of one-way valve mouth piece 100 directs air-flow in one direction (prevents reverse air-flow direction) through (a) conduit 101 in one-way valve mouth piece 100, (b) second bottom hollow neck 76 of second tube connector 70, (c) corrugated tubing 80, (d) first bottom hollow neck 66 of first tube connector 60 and mesh disc 40 sitting on first seat 61 in first tube connector 60, (e) inner and outer hollow chambers 33a and 33b of dual wall drug dispensing system 30 that create novel inner and outer air-flows in which drug substance 200 is concentrated within and propelled by the inner air-flow and the outer air-flow surrounds the inner air-flow to concentrate drug substance 200 within inner air-flow as it exits top end portion 31 of inner hollow chamber 33a and top 25 of through-hole 24 (not shown) of nose guard 20, so that drug substance 200, when breath-powered, is propelled from the novel, single-directional insufflator or breath-powered nasal device 10 meshmeshmeshmesh within a novel and unique double air-flow in a vertical, somewhat tight or confined and narrow, concentrated plume in high drug concentration, i.e., the inner air-flow, deep into the superior nasal cavity for effective distribution into the olfactory region and the mucosa innervated by the trigeminal nerve and for direct diffusion therefrom into the brain that is driven by the blown air through the novel, the single-directional insufflator or breath-powered nasal device 10.

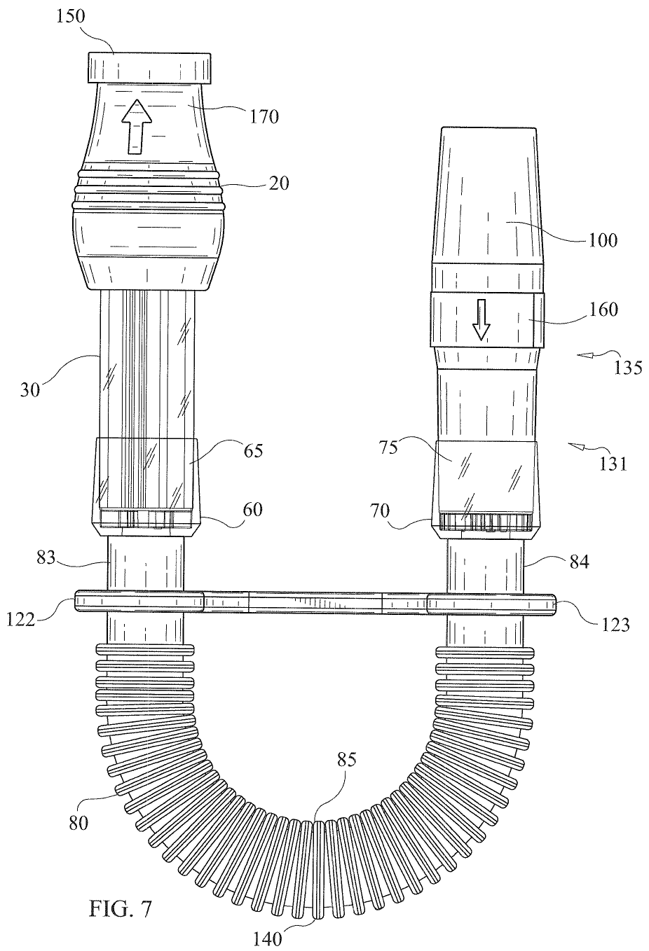

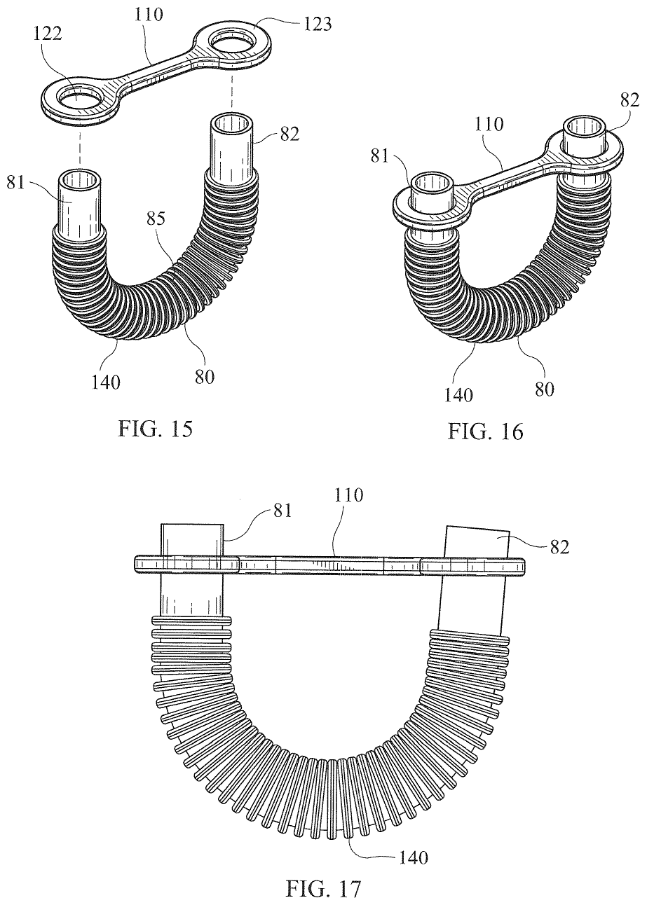

As depicted in FIGS. 7-8, a bridge connector 110 connects one side 131 of the novel, the single-directional insufflator or breath-powered nasal device 10 comprising the one-way valve mouth piece 100 and the second tube connector 70 to the other side 131 of the novel, the single-directional insufflator or breath-powered nasal device 10 comprising the nose guard 20, the dual wall drug dispensing system 30, the mesh disc 40 and the first tube connector 60 to maintain breath-powered insufflator or breath-powered nasal device 10 of the present invention in a “U-shape”configuration 140. Bridge connector 110 comprises bridge connector 110 sandwiched between a first hollow ring 122 and a second hollow ring 123 for sliding over the first hollow cuff 81 and second hollow cuff 82 of corrugated tubing 80. See FIGS. 15-16 and 18-19.

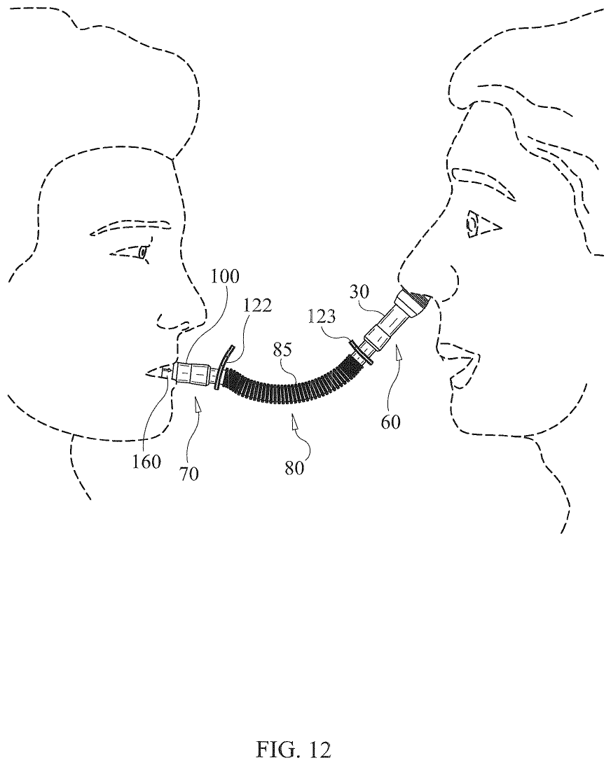

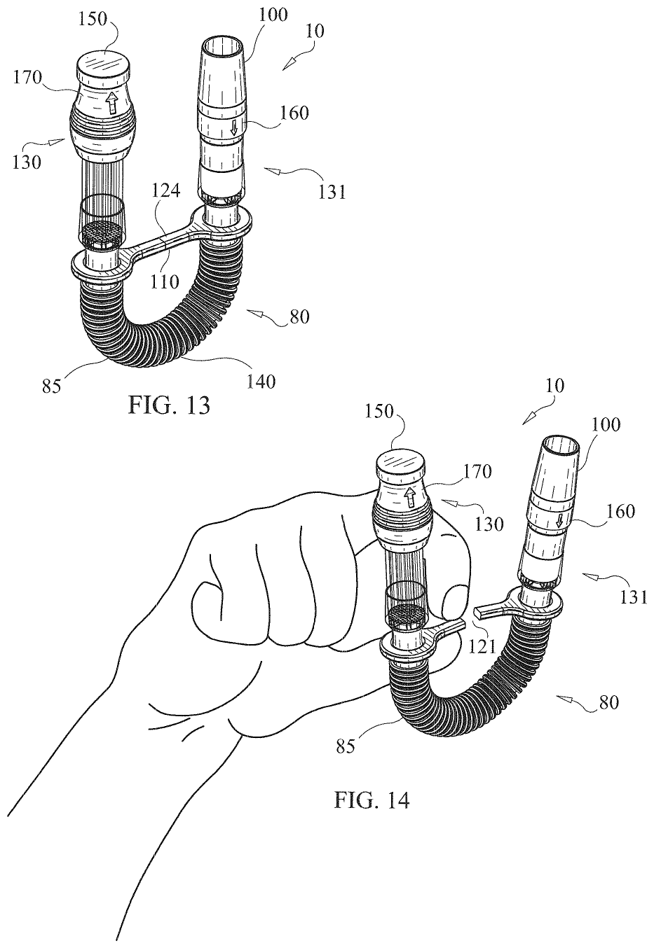

Alternatively, and as shown FIGS. 13-14, bridge connector 110 affixed to single-directional insufflator or breath-powered nasal device 10, illustrated in front view 135 with nose guard side 130 and one-way valve mouth piece side 131, may be designed with a perforation or score line 124 or any other configuration that allows bridge connector 110 to be broken and separated into two individual pieces in the event sides 130 and 131 of breath-powered insufflator or breath-powered nasal device 10 need to be orientated or adjusted at a different angle for use by, for example, a concussed person, or a second person who is assisting a person who, for example, is a concussed person, who cannot self-administer the necessary blow force to provide sufficient airflow to effectively propel the drug substance from the single-directional insufflator or breath-powered nasal device 10 of the present invention to effectively treat the concussion with the drug substance, as shown in FIG. 12.

Dual wall drug dispensing system 30, i.e., Dual-walled tubing configuration, is a novel and unique delivery system for delivering one or more drug substances into the nasal cavity. The dual wall drug dispensing system 30 comprises a top end portion 31 and the bottom end portion 32 and two hollow chambers 33, an inner hollow chamber 33a and an outer hollow chamber 33b. The drug substance 200 is propelled through hollow inner chamber 33a when the breath-powered insufflator or breath-powered nasal device 10 of the present invention is breath propelled. The hollow outer chamber 33b, which surrounds the hollow inner chamber 33a, is divided into two, three, four or more separate chambers 34 by rib walls 35. Preferably, but not necessarily critical, hollow outer chamber 33b is divided into three separate chambers 34. In addition to providing multiple hollow chambers 34, rib walls 35 impart integrity, structure and strength to and support for dual wall drug dispensing system 30.

While the present invention contemplates dual chamber 34 comprised of inner hollow chamber 33a and outer hollow chamber 33b of the dual wall drug dispensing system 30, i.e., Dual-walled tubing configuration, can be of any suitable dimensions necessary to perform effectively the objectives of the present invention, it is preferred that inner hollow chamber 33a can accommodate up to about 120 mg or more and any amount there between of drug substance in dry powder or other form and its outer wall diameter 331 can range from, for example, about 3 mm to about 7 mm or more or less and its inner wall diameter 332 can range from about 2 mm to about 5 mm or more or less, and outer wall diameter 333 can range from, for example, about 9 mm to about 13 mm or more or less and its inner wall diameter 334 can range from about 7 mm to about 12 mm or more or less.

Preferably, and by way of example, as shown in FIG. 29, one inner hollow chamber 33a contemplated by the present invention is dimensioned with an outer wall 331 diameter of about 5 mm and an inner wall 332 diameter of about 3 mm creating an internal gap 37 diameter of inner hollow camber 33a of about 3 mm. While these exemplary diameter dimensions generate about a 3 mm internal gap 37 between outer wall 333 of inner hollow chamber 33a and inner wall 334 of outer hollow chamber 33b, the present invention also contemplates inner hollow chambers 33a designed with an internal gap 37 diameter of up to about 1 mm to about 4 mm or greater. Thus, in accordance with the present invention, and preferably, internal gap 37 diameters may range from about 1 mm to about 4 mm or more, including internal gap 37 diameters of about 1 mm, about 1.5 mm, about 2 mm, about 2.5 mm, about 3 mm, about 3.5 mm and about 4 mm and dimension sizes there between, so that nose guard 20 can of a size that it allows nose guard 20 can be sized to snuggly accommodate any size nostril as discussed herein before.

While the thicknesses of outer wall 331 and inner wall 332 of inner hollow chamber 33a and outer wall 333 and inner wall 334 of outer hollow chamber 33b may be of any suitable thickness, it is preferred that the actual thickness of walls, 331, 332 and 333, 334 of the dual wall drug dispensing system 30, i.e., dual-walled tubing configuration, is about 2 mm thick.

To load the drug substance 200, when in a powdered, spray-dried format, into a novel single-directional insufflator or breath-powered nasal device 10 of the present invention, a sterilized funnel (not shown) is used to load the powdered drug substance 200 specifically within inner hollow chamber 33a of dual wall drug dispensing system 30. The mesh disc 40, which is positioned at bottom end portion 32 of inner hollow chamber 33a and on first seat 61 of first tube connector as shown in FIGS. 9-10 and 23, is what the loaded powdered drug substance 200 first encounters and against which it rest once fully loaded. Because the pore size of mesh disc 40 is smaller than the particle size of the powdered drug substance, the powdered drug substance cannot fall through mesh disc 40. The pores throughout mesh disc 40, however, permit the blown airflow to penetrate up and through mesh disc 40 to carry and propel powdered drug substance 200 through the inner hollow chamber 33a of dual wall drug dispensing system 30, i.e., the double walled-tube configuration, and top 25 of through-hole 24 and out of nose guard 20 deeply into the nasal cavity. It should be understood that if a liquid, suspension, gel or the like is loaded into dual wall drug dispensing system 30, i.e., the double walled-tube configuration, a different mesh disc 40 that prevents leakage or seepage there through may be warranted.

Preferably, the particle size distribution of powdered, spray-dried, drug substance 200 is principally in the range of about 1 μm to about 10 μm or more. In one embodiment, the powdered, spray-dried, drug substance 200 contains a medicament, particularly for the treatment of a nasal condition. Notwithstanding the preferred particle size, the present invention contemplates any effective particle size distribution of a powdered, spray-dried, drug substance that will accomplish the objectives of the present invention, including larger particles, or a smaller fraction of larger particles, typically in the range of about 10 μm to about 30 μm, and preferably in the range of about 20 μm to about 30 μm.

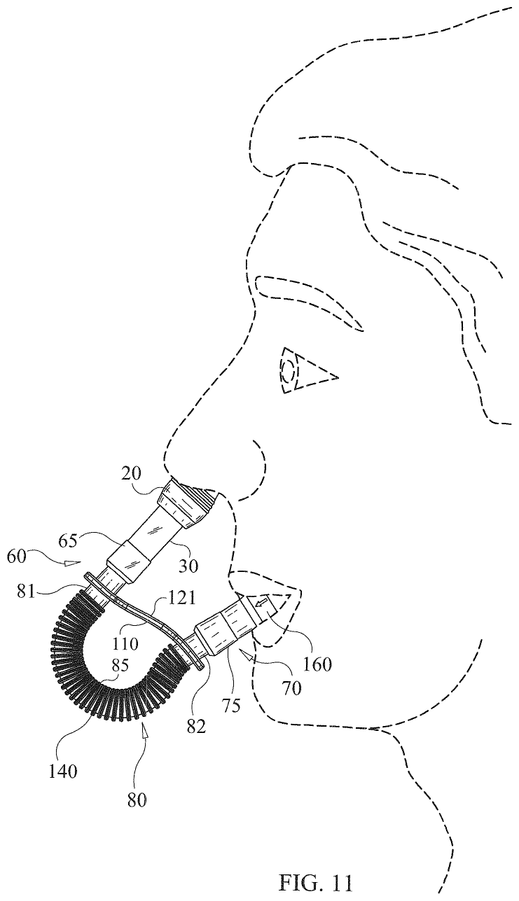

Turning now to operation, when a person blows or exhales through mouth piece 100 of the single-directional insufflator or breath-powered nasal device 10 of the present invention, as shown in FIGS. 11 and 12, the blown air flows through both hollow chambers 33a and 33b of dual wall drug dispensing system 30, i.e., the double walled-tube configuration, simultaneous and with essentially the same force expelling the drug substance loaded into inner hollow chamber 33a from nose guard 20. The air-flow pressure exiting from outer hollow chamber 33b surrounds the airflow burdened with high drug substance concentration exiting from inner hollow chamber 33a forcing the drug substance loaded air-flow to travel at least initially in a vertical, somewhat tight or confined and narrow, concentrated plume carrying high drug substance concentration into the deepest part of the superior nasal cavity, namely, the olfactory region and nerve and the mucosa innervated by the trigeminal nerve for direct diffusion therefrom into the brain. Thus, novel dual wall drug system 30, i.e., a dual wall-tube configuration, uniquely and surprisingly delivers high drug substance concentration to the targeted olfactory region, even the mucosa innervated by the trigeminal nerve, for direct diffusion into the brain for effective treatment of CNS injury, disease and/or disorders, including the brain, the brain stem, and the spinal cord and, in particular TBI, especially concussion.

While it should now be appreciated that the unique design of the single-directional insufflator or breath-powered nasal device 10 of the present invention uniquely targets drug deposition at and into the olfactory region and the trigeminal nerve mucosa for direct diffusion into the brain, the initial flow pattern of the exiting drug substance loaded airflow may fan out to some extent causing some drug substance to be deposited on other parts of the nasal cavity. It should be further appreciated that, while it is preferred to load only the inner hollow chamber 33a with drug substance 200, different or same drug substance(s) may be loaded into the two or more separate chambers 34 of outer hollow chamber 33b for deposition of drug substance(s) throughout the nasal cavity to treat CNS and nasal cavity and other nasal disorders treated via nasal application.

Thus, the present invention is uniquely suited to treat local, systemic and CNS injuries and/or disorders. It should be understood, however, that the single-directional insufflator or breath-powered nasal device 10 of the present invention is preferably a single use, disposable device especially designed to treat TBI, preferably mild, moderate and severe TBI, more preferably, mild to moderate TBI, even more preferably mild TBI, and most preferably, concussion. An example of drug substances suitable for use in combination with the single-directional insufflator or breath-powered nasal device 10 are C-20 steroid compounds in dry powder, spray-dried form, as disclosed and described in U.S. Patent Publication No. 2016/0168190, entitled “C-20 steroid compounds, compositions and uses thereof,” and WO2016/044559A1, entitled “C-20 steroid compounds, compositions and uses thereof to treat traumatic brain injury (TBI), including concussions,” each of which is incorporated herein by reference in its entirety as if it was fully set forth herein. One preferred C-20 steroid compound that is contemplated by the present invention is ent-19-norprogesterone in dry-powder, spray-dried form. Ent-19-norprogesterone has a molecular formula of C₂₀H₂₈O₂ and a molar mass of 300.435 g/mol. One chemical name for ent-19-norprogesterone is ent-19-norpregn-4-ene-3,20-dione. The chemical structure of ent-19-norprogesterone is as follows:

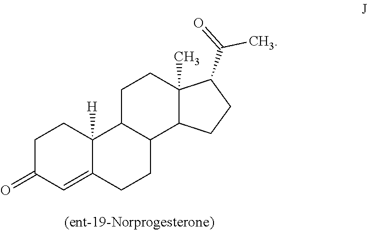 [SMILES: CC(=O)[C@@H]1CCC2C3CCC4=CC(=O)CC[C@H]4C3CC[C@]21C]

ent-19-norprogesterone is a fully synthetic, non-naturally, occurring neurosteroid. In preclinical studies, ent-19-norprogesterone has demonstrated equivalent, if not superior, neuroprotective effects compared to related neurosteroids. Animal models of concussion demonstrated that ent-19-norprogesterone reduces the behavioral pathology associated with brain injury symptoms such as memory impairment, anxiety, and motor/sensory performance. Additionally, ent-19-norprogesterone is lipophilic and can easily cross the blood-brain barrier to rapidly eliminate or at least reduce swelling, oxidative stress and inflammation in the brain while the healing process from TBI, e.g., concussion, attempts to restore proper blood flow and heal damaged or affected brain and/or other tissue.

When ent-19-norprogesterone is the drug substance of choice to treat TBI, especially concussion, inner hollow chamber 33a is pre-loaded in accordance with the present invention with preferably about 100 mg of drug substance ent-19-norprogesterone, wherein the ent-19-norprogesterone drug substance comprises about 8 mg and the pharmaceutical acceptable excipient, hydroxypropyl-ß-cyclodextrin, comprises about 92 mg. This 19-norprogesterone formulation is preferably in dry-powder, spray-dried format and can be administered up to four or more times a day for as long as necessary in accordance with a prescribed treatment regimen to treat, for example, a patient suffering with TBI, especially a concussion. To administer ent-19-norprogesterone drug substance preloaded into inner hollow chamber 33a of the dual wall drug dispensing system 30 of the single-directional insufflator or breath-powered nasal device 10 of the present invention, the concussed or TBI injured person inserts nose guard 20 into one nasal vestibule or nostril until it is snug-fitting and then inserts mouth piece 100 between his/her lips. Once the single-directional insufflator or breath-powered nasal device 10 of the present invention is properly fitted in the nostril and between the lips of the concussed or TBI injured person, the concussed or TBI injured patient blows/exhales through mouth piece 100: (a) to close the velum or soft palate to isolate the nasal cavity to prevent the ent-19-norprogesterone drug substance from being inhaled or passing into the digestive system and therefore reducing systemic side effects; and (b) to propel the ent-19-norprogesterone drug substance in the inner hollow chamber 33a from the nose guard 20 with sufficient airflow force and pressure, that concentrated ent-19-norprogesterone drug substance is deposited onto and distributed throughout the olfactory region and the trigeminal nerve mucosa for direct diffusion into the brain for effective treatment of the concussion or TBI within minutes of nasal application. Importantly, one-way valve mouth piece 100 prevents reverse air-flow and the closed soft palate will trap the concentrated ent-19-norprogesterone drug in the nasal cavity allowing it to stick to the superior nasal membranes for diffusion directly into the brain by way of the olfactory nerve and the trigeminal nerve mucosa.

This very advantageous and unique nasal delivery is accomplished by the novel dual wall drug dispensing system 30, i.e., the double walled-tube configuration, of the single-directional insufflator or breath-powered nasal device 10 of the present invention through the use of the unique dual air-flow combination of (i) a first air-flow expelled from the inner hollow chamber 33a carrying the ent-19-norprogesterone drug substance, and (ii) a second air-flow expelled simultaneously from the outer hollow chamber 33b to create air-flow pressure that surrounds the first expelled air-flow, so that the first expelled air flow forms and travels initially in a vertical, somewhat tight or confined and narrow, concentrated plume that drives a concentrated drug substance, e.g., ent-19-norprogesterone drug substance, to the targeted olfactory region, including the olfactory nerve, and the trigeminal nerve mucosa for direct diffusion into the brain and for effective treatment of the concussion or TBI within minutes of nasal application.

While single-directional insufflator or breath-powered nasal device 10 of the present invention is simple in construction, the operation of the device does provide for the effective delivery of drug substances, in particular one of powder containing the drug substance, to the posterior region of the nasal airway, since the powdered drug substance is delivered in a single-direction deep into the nasal cavity and the air-flow into the nasal cavity is through the same opening, namely, the respective nostril, with the closed posterior region of the respective nasal cavity acting as a pressure reflecting surface which causes the exhaled air to adequately reach the posterior region of the respective nasal cavity for deposition into the olfactory region and the trigeminal nerve mucosa for rapid diffusion into the brain for the treatment of nasal and/or CNS injury, disease or disorder, especially brain injury, such as TBI, including concussion. Further, in providing a short explosive burst of air-flow into one of the nasal cavities, it is now possible to achieve a sustained single-directional air-flow deep within the nasal cavity of high concentrated powdered drug substance, which is believed to be necessary to deliver a powdered drug substance effectively to the posterior region of the nasal airway for deposition into the olfactory region and the trigeminal nerve mucosa for rapid diffusion into the brain.

As contemplated by the present invention, any suitable materials may be used for the individual parts or components of the single-directional insufflator or breath-powered nasal devices 10 of the present invention, VESTAMID ML21 is an example of a selected material for plastic parts, e.g., dual wall drug dispensing system 30 and tube connectors 60 and 70. ABS is an exemplary material for mouth piece 100 and one-way valve 50 (not shown). PTFE is an exemplary material for corrugated tubing 80. Silicone HD-150A is exemplary material for nose guard 20, bridge connector 110 and seal cap 150, i.e., top cover.

In accordance with one embodiment of the present invention, the length of the dual wall drug dispensing system 30, i.e., the double walled-tube configuration, is about 2.00 inches and the length of the corrugated tube is about 4 inches. Total length of such an exemplary device is about 8 inches. When in U-shape configuration, the maximum length of such device is about 4 inches from the bottom of the corrugated material to the top of the nose guard 20 and the width is about 2.2 inches. The width of the corrugated material from side view is about 0.45 inches and the widest width of nose guard 20 is about 0.75 inches, as shown in FIG. 38, and the widest width of one-way valve mouth piece 100 is about 0.5 inches.

It should be further understood that the above summary of the present invention is not intended to limit or describe each and every possible embodiment or implementation of the present invention, but contemplates such possible embodiments and implementations. The description, however, does exemplify illustrative embodiments. In several places throughout the specification, guidance is provided through examples, which examples can be used in various combinations. In each instance, the examples serve only as representative groups and should not be interpreted as exclusive examples.

### DESCRIPTION OF THE FIGS.

The structure, operation and advantages of this invention will become further apparent upon consideration of the following description, taken in conjunction with the accompanying Figs., wherein:

FIG. 1 is a front assembled elevational angled front view 135 of a single-directional insufflator or breath-powered nasal device 10, in accordance with an embodiment of the of the present invention;

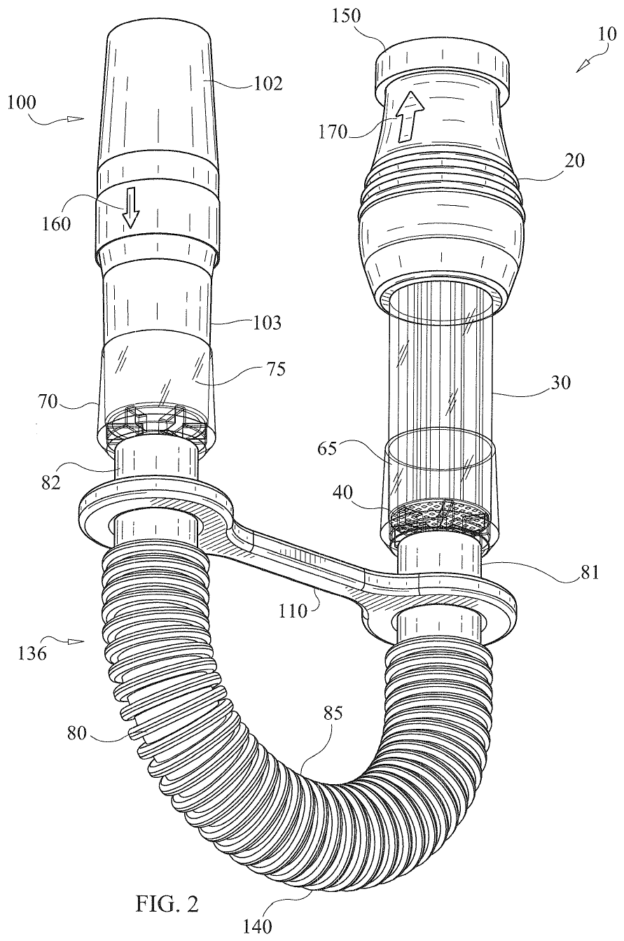

FIG. 2 is a rear assembled elevational angled back view 136 of a single-directional insufflator or breath-powered nasal device 10, in accordance with an embodiment of the of the present invention;

FIG. 3 is a top view 850 of a corrugated tube 80 showing corrugated middle section 85 and bridge connector 110, in accordance with an embodiment of the of the present invention;

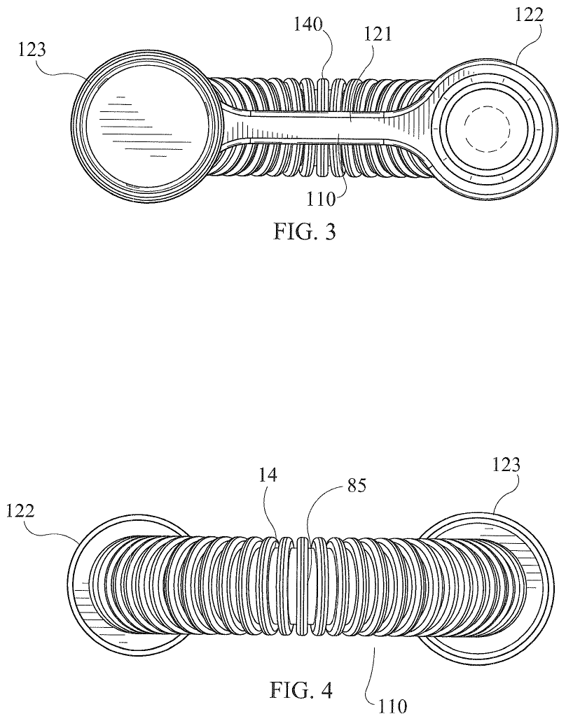

FIG. 4 is a bottom view 860 of a corrugated tube 80 showing corrugated middle section 85, in accordance with an embodiment of the of the present invention;

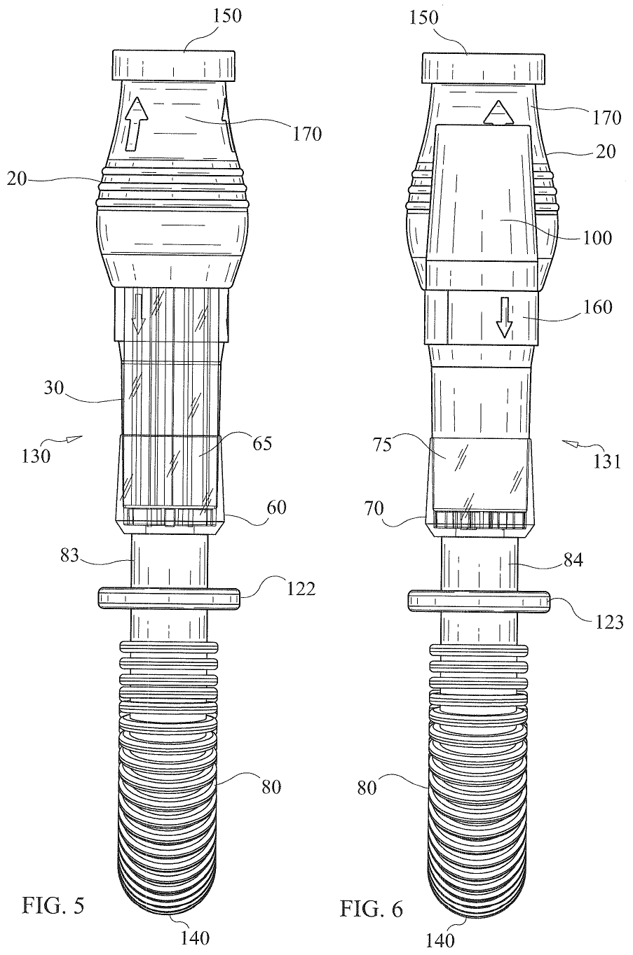

FIG. 5 is an assembled elevational view of nose guard side 130 of the single-directional insufflator or breath-powered nasal device, in accordance with an embodiment of the of the present invention;

FIG. 6 is an assembled elevational view of the one-way valve mouth piece side 131 of the single-directional insufflator or breath-powered nasal device, in accordance with an embodiment of the of the present invention;

FIG. 7 is a right mouth piece assembled elevational view of a single-directional insufflator or breath-powered nasal device, in accordance with an embodiment of the of the present invention;

FIG. 8 is a left mouth piece assembled elevational view of a single-directional insufflator or breath-powered nasal device, in accordance with an embodiment of the of the present invention;

FIG. 9 is a right mouth piece front unassembled exploded view of a single-directional insufflator or breath-powered nasal device, in accordance with an embodiment of the of the present invention;

FIG. 10 is a left mouth piece front unassembled exploded view of a single-directional insufflator or breath-powered nasal device, in accordance with an embodiment of the of the present invention;

FIG. 11 schematically illustrates a single-directional insufflator or breath-powered nasal device of FIGS. 1-2 and 4-8 inserted into a nasal cavity of a subject for operation, in accordance with an embodiment of the of the present invention;

FIG. 12 schematically illustrates a nose guard of an alternative embodiment of a single-directional insufflator or breath-powered nasal device as shown in FIGS. 13 and 14 inserted into a nasal cavity of a first subject to be treated with a drug substance and a mouth piece of the alternative embodiment as shown in FIGS. 13 and 14 inserted between the lips of a second subject for operation of the alternative embodiment by the second subject as shown in FIGS. 13 and 14, in accordance with an embodiment of the of the present invention;

FIG. 13 is a front, right mouth piece, assembled elevational angled view of an alternative embodiment of a single-directional insufflator or breath-powered nasal device herein wherein the bridge connector is designed with a perforated or score line for breaking the bridge connector, in accordance with an embodiment of the of the present invention;

FIG. 14 is a front, right mouth piece, assembled elevational angled view of an alternative embodiment of a single-directional insufflator or breath-powered nasal device herein wherein the bridge connector is broken in two pieces at the perforated or score line for breaking the bridge connector, as shown in FIG. 13, in accordance with an embodiment of the of the present invention;

FIG. 15 schematically illustrates assembling bridge connector 110 to corrugated tubing 80, in accordance with an embodiment of the of the present invention;

FIG. 16 schematically illustrates a perspective view of bridge connector 110 and corrugated tubing 80 assembled in accordance with an embodiment of the of the present invention;

FIG. 17 schematically illustrates a front view of bridge connector 110 and corrugated tubing 80 assembled in accordance with an embodiment of the of the present invention;

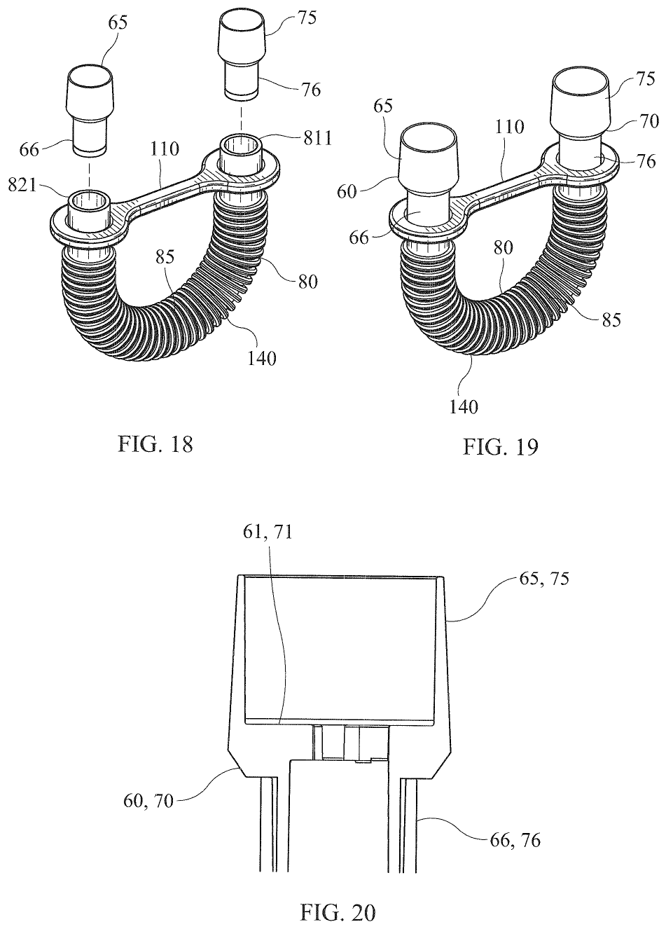

FIG. 18 schematically illustrates assembling first tube connector 60 and second tube connector 70 to corrugated tubing 80 assembled with bridge connector 110, as shown in FIGS. 16 and 17, in accordance with an embodiment of the of the present invention;

FIG. 19 schematically illustrates a perspective view of first tube connector 60 and second tube connector 70 assembled with corrugated tubing 80 assembled with bridge connector 110, as shown in FIGS. 16 and 17, in accordance with an embodiment of the of the present invention;

FIG. 20 schematically illustrates a cross-sectional view of a tube connector, in accordance with an embodiment of the of the present invention;

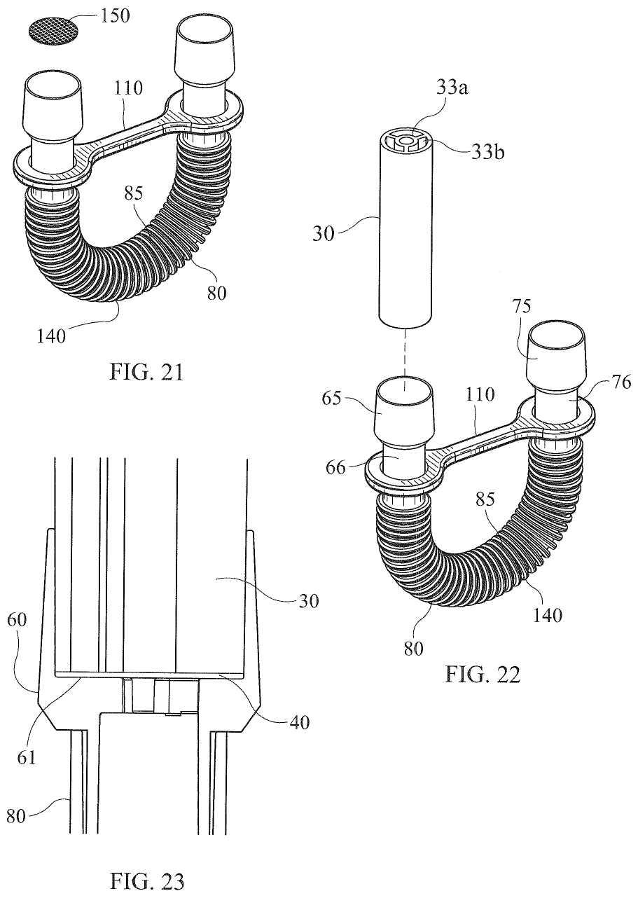

FIG. 21 schematically illustrates inserting a mesh disc 40 into first tube connector 60, wherein first tube connector 60 and second tube connector 70 are assembled with corrugated tubing 80 assembled with bridge connector 110, as shown in FIGS. 16 and 17, in accordance with an embodiment of the of the present invention;

FIG. 22 schematically illustrates inserting a dual wall drug dispensing system 30, i.e., a double-walled tube configuration, into first tube connector 60, wherein mesh disc 40 is inserted into first tube connector 60 and first tube connector 60 and second tube connector 70 are assembled with corrugated tubing 80 assembled with bridge connector 110, as shown in FIGS. 16, 17, 19 and 21, in accordance with an embodiment of the of the present invention;

FIG. 23 schematically illustrates an exploded cross-sectional view of assembled first tube connector 60, wherein mesh disc 40 and dual wall drug dispensing system 30, i.e., a double-walled tube configuration, are assembled within first tube connector 60 and mesh disc 40 is sitting on first seat 61 of first tube connector 60 and first top hollow collar 65 of first tube connector 60 is press-fit into first hollow cuff 81 of corrugated tubing 80, in accordance with an embodiment of the of the present invention;

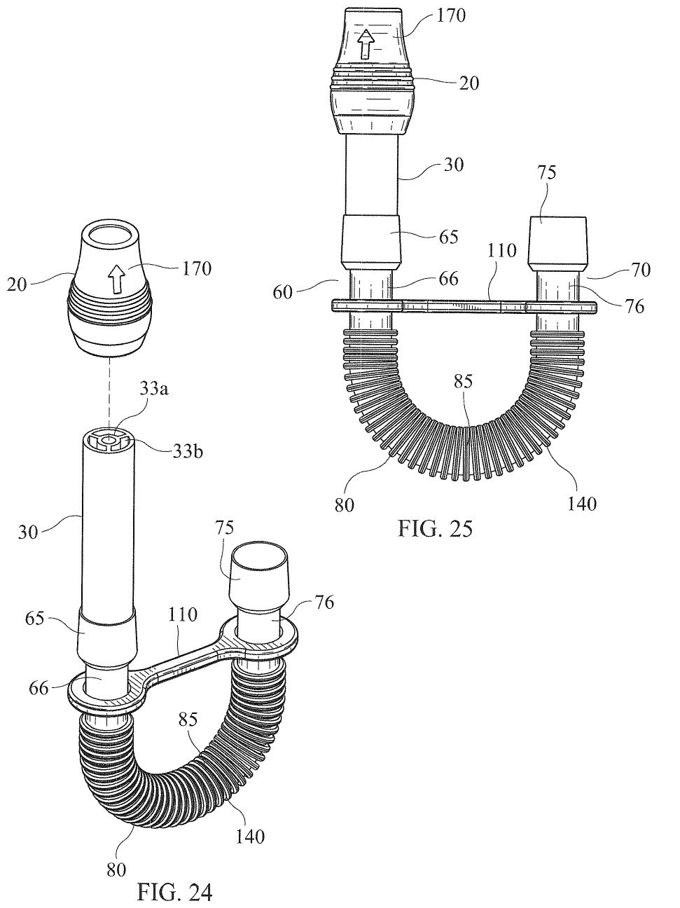

FIG. 24 schematically illustrates inserting top end portion 31 of dual wall drug dispensing system 30, i.e., the double walled-tube configuration, into bottom 26 of through-hole 24 of nose guard 20, wherein mesh disc 40 is assembled within first tube connector 60 and first tube connector 60 and second tube connector 70 are assembled with corrugated tubing 80 assembled with bridge connector 110, as shown in FIGS. 16, 17, 19, 21 and 22, in accordance with an embodiment of the of the present invention;

FIG. 25 schematically illustrates a front view of assembled nose guard 20, first tube connector 60, mesh disc 40 (not shown), dual wall drug dispensing system 30, i.e., a double-walled tube configuration, first tube connector 60 and second tube connector 70, bride connector 110 and corrugated tubing 80, in accordance with an embodiment of the of the present invention;

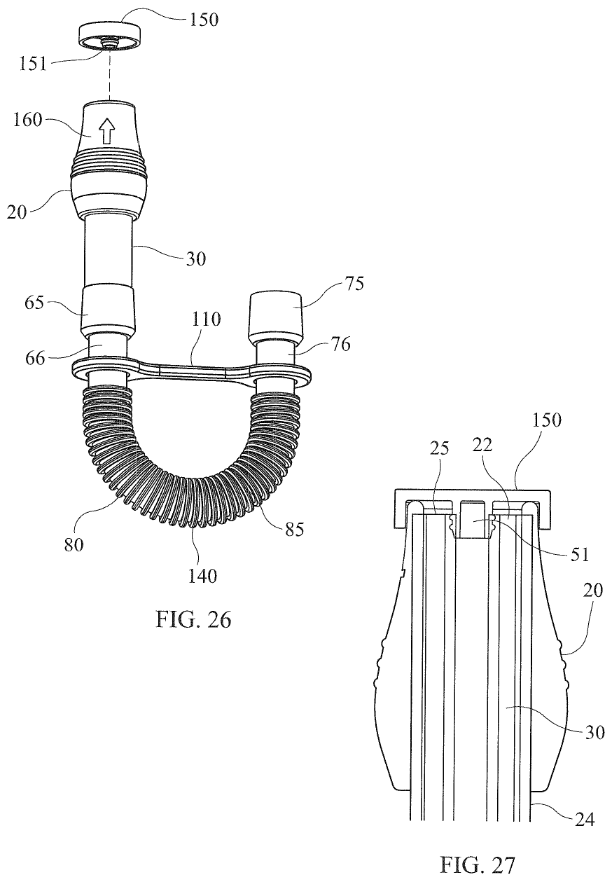

FIG. 26 schematically illustrates placing seal cap 150 with seal cap plug 151 into top 25 (not shown) of through-hole 24 (not shown) over nose guard 20 of assembled nose guard 20, first tube connector 60, mesh disc 40 (not shown), dual wall drug dispensing system 30, i.e., a double-walled tube configuration, first tube connector 60 and second tube connector 70, bride connector 110 and corrugated tubing 80, in accordance with an embodiment of the of the present invention;

FIG. 27 schematically illustrates an exploded cross-sectional view of nose guard 20, wherein dual wall drug dispensing system 30, i.e., the double walled-tube, is inserted through through-hole 24 of nose guard 20, top end portion 31 of dual wall drug dispensing system 30, i.e., a double-walled tube configuration, is seated against nose guard seat 22 of nose guard 20 and seal cap 150 is positioned over nose guard 20 with nose guard stem 151 is inserted through top 25 and into through-hole 24 of nose guard 20, in accordance with an embodiment of the of the present invention;

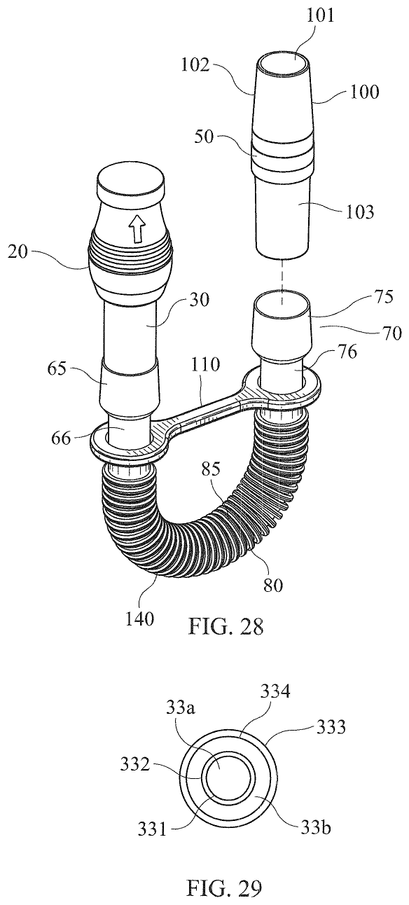

FIG. 28 schematically illustrates inserting bottom section 102 of one-way valve mouth piece 100 into of dual wall drug dispensing system 30, i.e., the double walled-tube, into second top hollow collar 75 of second tube connector 70, assembled seal cap 150, nose guard 20, first tube connector 60, mesh disc 40 (not shown), dual wall drug dispensing system 30, i.e., a double-walled tube configuration, first tube connector 60 and second tube connector 70, bride connector 110 and corrugated tubing 80, in accordance with an embodiment of the of the present invention;

FIG. 29 is an exploded cross-sectional view of inner hollow chamber 33a, having outer wall 331 and inner wall 332, and outer hollow chamber 33b having outer wall 333 and inner wall 334 of dual wall drug dispensing system 30, i.e., a double-walled tube configuration, illustrating one size tube diameters, wherein, outer hollow chamber 33b having outer wall 333 diameter of 11 mm and inner wall 334 diameter of 9 mm and inner hollow chamber 33a having outer wall 331 diameter of 6 mm and inner wall 332 diameter of 4 mm with internal gap 36 of 1.5 mm and an inner hollow chamber 33a having an internal diameter of 4 mm. Illustrations of alternative size tube diameters are: (a) outer hollow chamber 33b having outer wall 333 diameter of 10 mm and inner wall 334 diameter of 8 mm and inner hollow chamber 33a having outer wall 331 diameter of 6 mm and inner wall 332 diameter of 4 mm with internal gap 36 of 1 mm and an inner hollow chamber 33a having an internal diameter of 4 mm; (b) outer hollow chamber 33b having outer wall 333 diameter of 11 mm and inner wall 334 diameter of 9 mm and inner hollow chamber 33a having outer wall 331 diameter of 5 mm and inner wall 332 diameter of 3 mm with internal gap 36 of 2 mm and an inner hollow chamber 33a having an internal diameter of 3 mm; and (c) outer hollow chamber 33b having outer wall 333 diameter of 13 mm and inner wall 334 diameter of 11 mm and inner hollow chamber 33a having outer wall 331 diameter of 5 mm and inner wall 332 diameter of 3 mm with an internal gap 36 of 3 mm and an inner hollow chamber 33a having an internal diameter of 3 mm, in accordance with various embodiments of the of the present invention;

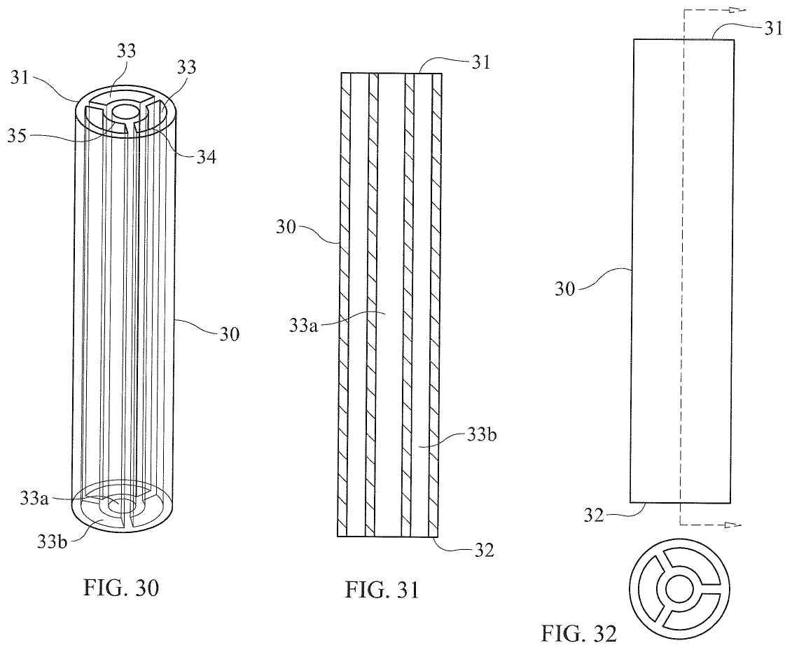

FIG. 30 is a perspective view of dual wall drug dispensing system 30, i.e., a double-walled tube configuration, bottom end portion 32, internal gap 36 and the two hollow chambers 33, i.e., the inner hollow chamber 33a and the outer hollow chamber 33b, wherein the outer hollow chamber 33b is divided into three separate chambers 34 by rib walls 35, in accordance with an embodiment of the of the present invention;

FIG. 31 is a cross-sectional view of dual wall drug dispensing system 30, i.e., a double-walled tube configuration, as shown in FIGS. 9, 10, 19 and 30, in accordance with an embodiment of the of the present invention;

FIG. 32 is a cross-sectional view taken along line A of dual wall drug dispensing system 30, i.e., a double-walled tube configuration, as shown in FIGS. 9, 10, 19, 30 and 31, in accordance with an embodiment of the of the present invention;

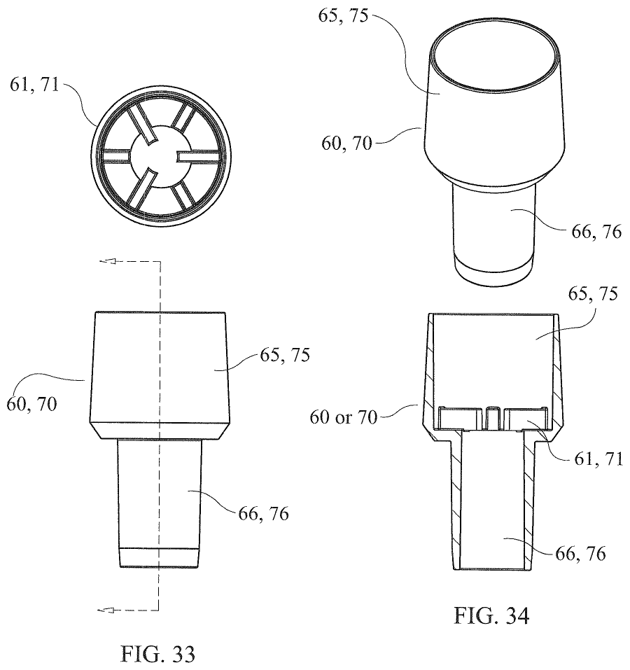

FIG. 33 is a cross-sectional view of tube connector 60 or 70 taken along line A, as shown in FIGS. 9, 10, 18, 23 and 31, in accordance with an embodiment of the of the present invention;

FIG. 34 is a cross-sectional view of tube connector 60 or 70, as shown in FIGS. 9, 10, 18, 23 and 31, in accordance with an embodiment of the of the present invention;

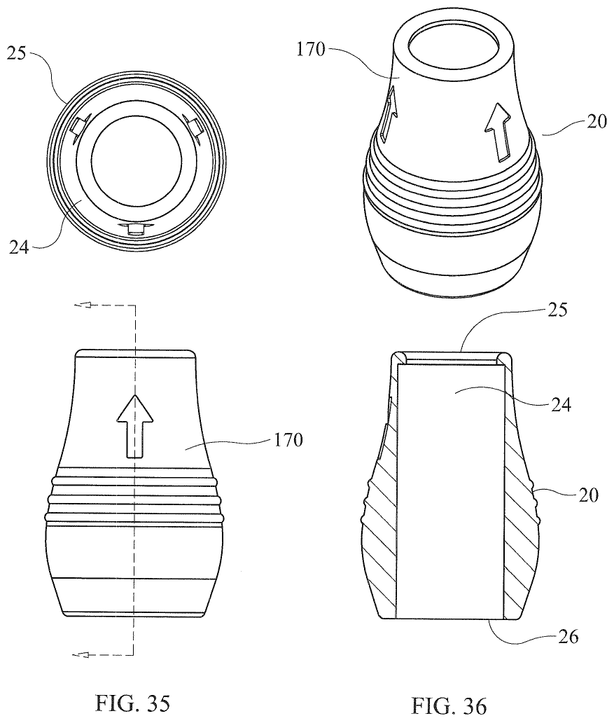

FIG. 35 is a cross-sectional view of nose guard 20 taken along line A depicting top 25 of through-hole 24, as shown in FIGS. 9, 10, 24, 25 and 27, in accordance with an embodiment of the of the present invention;

FIG. 36 schematically illustrates an alternative cross-sectional view of nose guard 20, depicting top 25 and bottom 26 of through-hole 24 and nose guard seat 22, as shown in FIGS. 9, 10, 24, 25, 27 and 35, in accordance with an embodiment of the of the present invention;

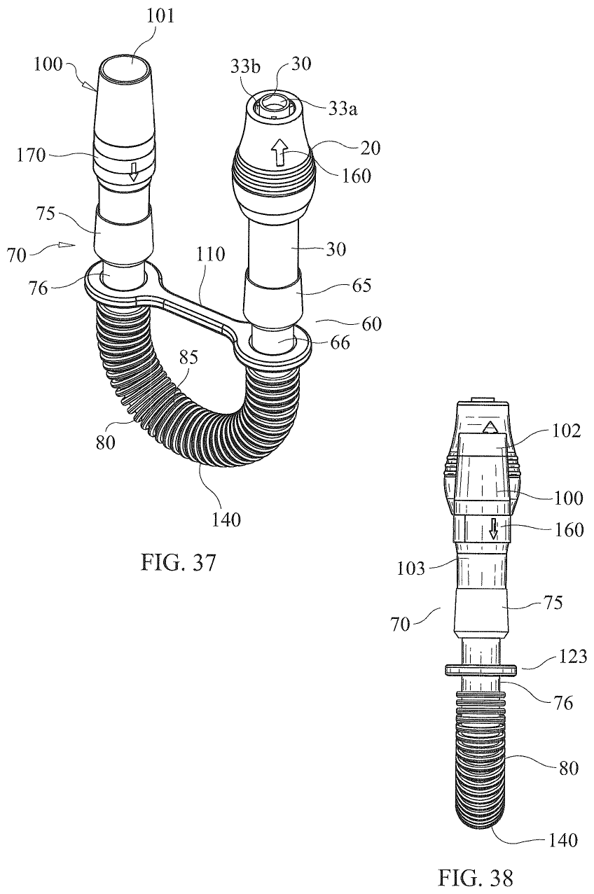

FIG. 37 schematically illustrates an assembled view of single-directional insufflator or breath-powered nasal device 10 depicting one-way valve mouth piece 100, first air-flow directional band 160, first tube connector 60, corrugated tubing 80, bridge connector 110, second tube connector 70, second air-flow directional band 170, dual wall drug dispensing system 30 and nose guard 20, in accordance with an embodiment of the of the present invention;

FIG. 36 schematically illustrates an alternative one-way valve mouth piece 131 view of and assembled single-directional insufflator or breath-powered nasal device 10 nose guard, as shown in FIG. 6, in accordance with an embodiment of the of the present invention;

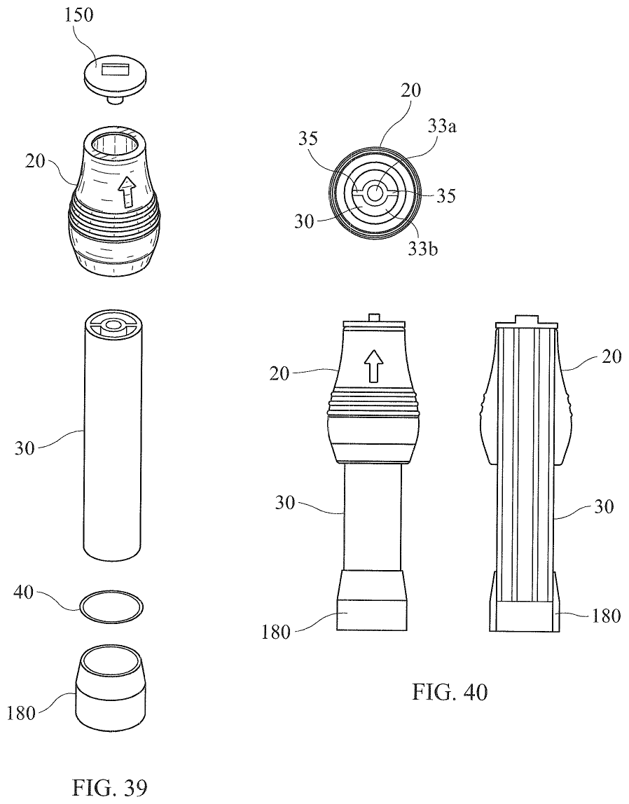

FIG. 39 schematically illustrates an unassembled view of seal cap 150, i.e., top plug, nose guard 20, one-way valve dual wall drug dispensing system 30, i.e., a double-walled tube configuration, mesh disc 40, and alternative connector 180 for connecting one-way dual wall drug dispensing system 30, i.e., the double walled-tube, and corrugated tubing 80, in accordance with an embodiment of the of the present invention;

FIG. 40 schematically illustrates an alternative cross-sectional view of seal cap 150, i.e., top plug, nose guard 20, and one-way valve dual wall drug dispensing system 30, i.e., a double-walled tube configuration, assembled with alternative connector 180, as shown in FIG. 39, in accordance with an embodiment of the of the present invention;

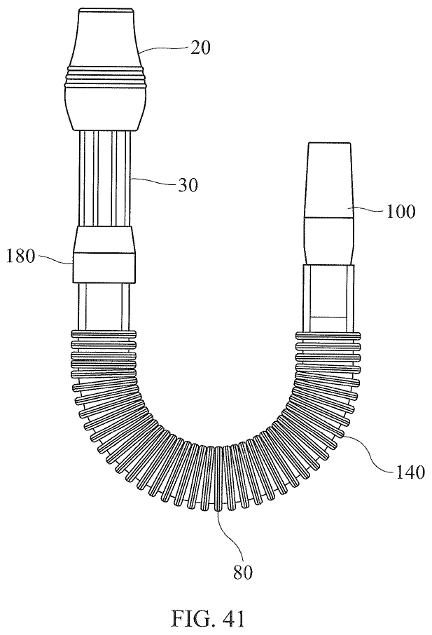

FIG. 41 schematically illustrates an assembled front view of nose guard 20, dual wall drug dispensing system 30, i.e., a double-walled tube configuration, alternative connector 180, corrugated tubing 80, and nose guard 20, in accordance with an embodiment of the of the present invention;

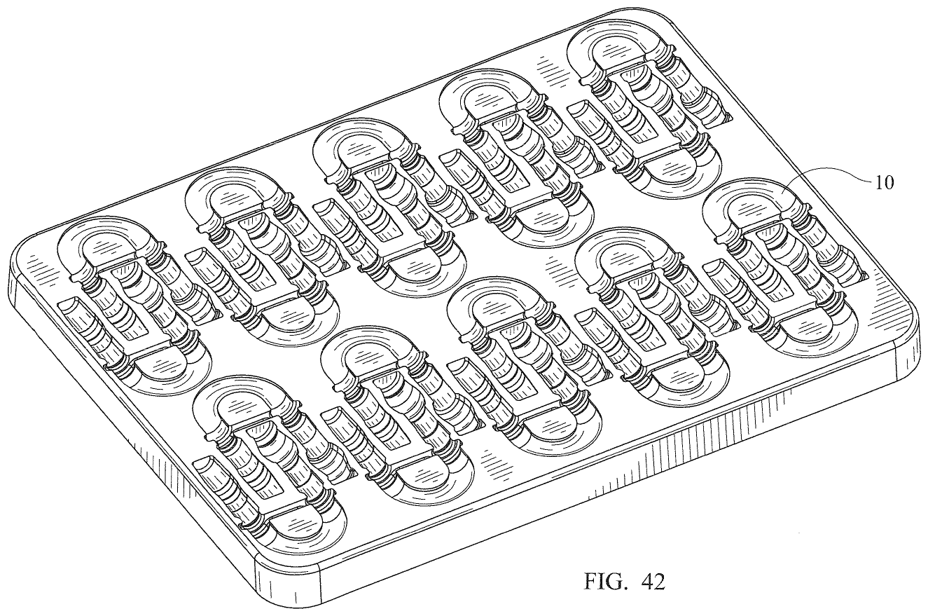

FIG. 42 schematically illustrates a top view of a tray packaged with twenty single-directional insufflator or breath-powered nasal fully assembled devices herein, wherein the 20 such devices are positioned in two rows of five pairs of such devices, wherein each such pair shows two such devices positioned in an interlocking position with respect to one another, in accordance with an embodiment of the of the present invention;

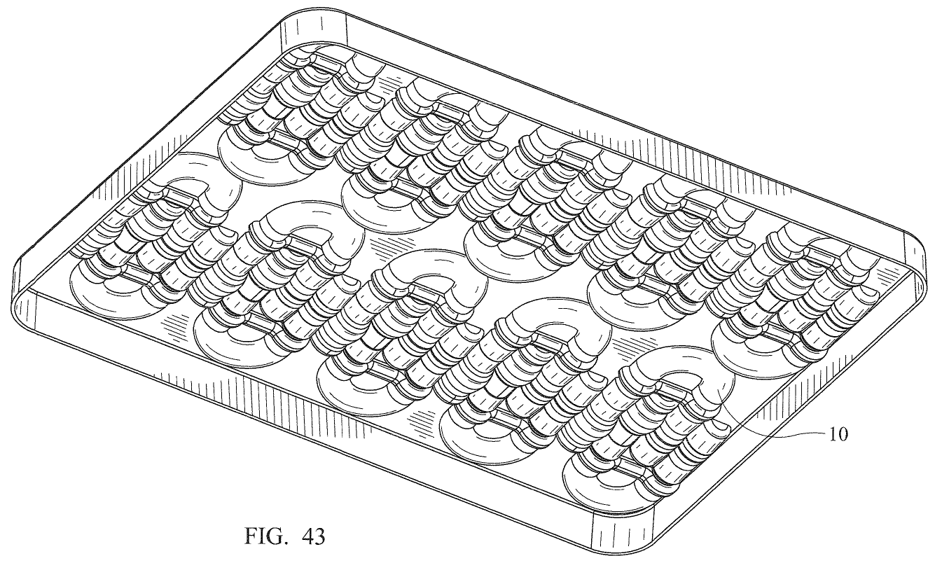

FIG. 43 schematically illustrates a side top view of a tray packaged exposing an internal side longitudinal wall with twenty single-directional insufflator or breath-powered nasal fully assembled devices herein, wherein the 20 such devices are positioned in two rows of five pairs of such devices, wherein each such pair shows two such devices positioned in an interlocking position with respect to one another, in accordance with an embodiment of the of the present invention;

FIG. 44 illustrates a screen shot comparing the width of a lactose powder spray between a single tube configuration and a dual wall drug dispensing system 30, i.e., a double-walled tube configuration, of the present invention in which the internal diameter of the inner tube of the double tube is about 4 mm and the gap between the inner and outer tubes of the double tube is either 1.5 mm or 1 mm to a single tube configuration, as compared to baseline, i.e., a single tube configuration, in accordance with an embodiment of the of the present invention;

FIG. 45 illustrates a screen shot comparing the width of a lactose powder spray between a single tube configuration and a dual wall drug dispensing system 30, i.e., a double-walled tube configuration, of the present invention in which the internal diameter of the inner tube of the double tube is about 3 mm and the gap between the inner and outer tubes of the double tube is either 2 mm or 3 mm to a single tube configuration, as compared to baseline, i.e., a single tube configuration, in accordance with an embodiment of the of the present invention;

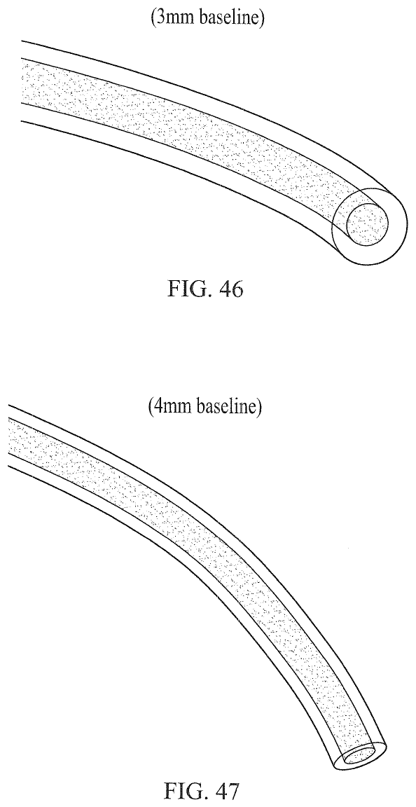

FIG. 46 is a screen shot of a single tube configuration with a 3 mm baseline following a lactose powder spray showing significant build up or accumulation of lactose powder on internal wall post-powder spray;

FIG. 47 is a screen shot of a single tube configuration with a 4 mm baseline following a lactose powder spray showing significant build up or accumulation of lactose powder on internal wall post-powder spray;

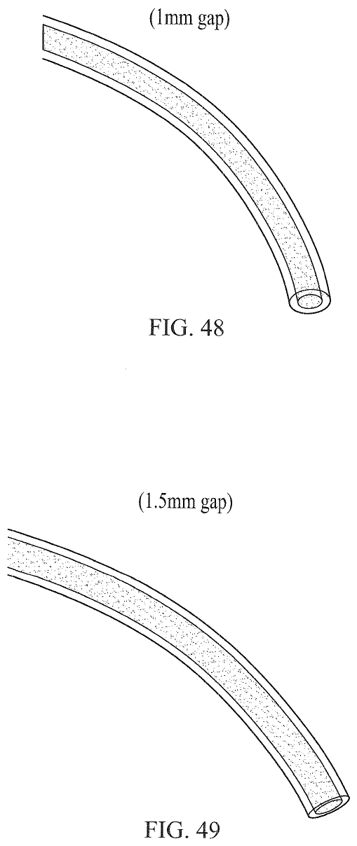

FIG. 48 is a screen shot of a dual wall drug dispensing system 30, i.e., a double-walled tube configuration, with a 1 mm gap following a lactose powder spray showing little build up or accumulation of lactose powder on inner wall 332 of inner hollow chamber 33a post-powder spray, as compared to the single tube configuration as shown in FIG. 45, in accordance with an embodiment of the of the present invention;

FIG. 49 is a screen shot of a dual wall drug dispensing system 30, i.e., a double-walled tube configuration, with a 1.5 mm gap following a lactose powder spray showing little build up or accumulation of lactose powder on inner wall 332 of inner hollow chamber 33a post-powder spray, as compared to the single tube configuration as shown in FIG. 45, in accordance with an embodiment of the of the present invention;

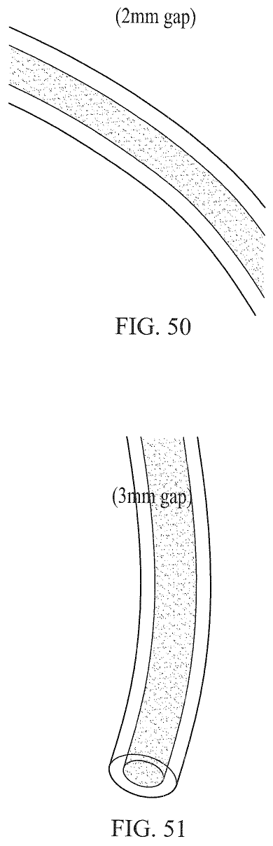

FIG. 50 is a screen shot of a dual wall drug dispensing system 30, i.e., a double-walled tube configuration, with a 2 mm gap following a lactose powder spray showing little build up or accumulation of lactose powder on inner wall 332 of inner hollow chamber 33a post-powder spray, as compared to the single tube configuration as shown in FIG. 45, in accordance with an embodiment of the of the present invention; and

FIG. 51 is a screen shot of a dual wall drug dispensing system 30, i.e., a double-walled tube configuration, with a 3 mm gap following a lactose powder spray showing little build up or accumulation of lactose powder on inner wall 332 of inner hollow chamber 33a post-powder spray, as compared to the single tube configuration as shown in FIG. 45.

### DESCRIPTION OF THE INVENTION

The present disclosure may be understood more readily by reference to the following detailed description and FIGS. of desired embodiments. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising,” as used herein and in a claim, means that the named elements or steps are essential, but other elements or steps may be added, present, comprehended or included. Thus, the term “comprising”, as used herein and in a claim, is synonymous with “including,” “comprehending,” “containing,” or “characterized by,” and therefore is an inclusive or open-ended claim and does not exclude additional, unrecited elements or steps. The term “comprising” should be construed to include the term “consisting of”, which allows the presence of only the named components/steps.

The term “consisting essentially of,” as used herein and in a claim herein, necessarily includes the recited elements or steps, but is open to unlisted elements or steps that do not materially affect the basic and material properties of the subject matter claimed, regardless of whether the subject matter is, e.g., a composition, compound, formulation, process or method.

The term “consists of,” as used herein and in a claim, excludes any element or step not expressly recited and thus is a closed claim.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 mg to 10 mg” is inclusive of the endpoints, 2 mg and 10 mg, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus up to 10% of the indicated number in the applicable or appropriate context. For example, “about 10%” may indicate a range of 9% to 11%, and “about 1” may mean from 0.9-1.1.

As used herein, a “composition” can be a single drug or combination drug (fixed-dose combination) combining at least the two agents, discussed infra, in a single dose. In another embodiment, the “composition” can include a combination of drugs or agents that are each administered or formulated separately or simultaneously. Similarly, a treatment can include administering to a patient a composition as a fixed-dose drug or agent combination, or it can include administering to a patient two or more separate agents or drugs for example, together, substantially together, concomitantly or in a predetermined sequence, frequency and time at their respective dosage strengths or amounts.

Actual dosage levels of a selective drug substance can be varied in order to achieve the effective and safe therapeutic response for a particular patient. The phrase “therapeutically effective amount” or “effective amount” means a sufficient amount of the drug substance to treat injury, diseases or disorders, at a reasonable benefit/risk ratio applicable to any medical treatment. It will be understood, however, that the total daily usage of the drug substances of the present disclosure will be decided by the attending physician or other care provider within the scope of sound medical judgment. If desired, the effective daily dose can be divided into multiple doses for purposes of administration; consequently, single dose drug substances may contain such amounts or submultiples thereof to make up the daily dose. The specific therapeutically effective dose level for any particular patient will depend upon a variety of factors, including the injury, disease or disorder being treated and the severity of the injury, disease or disorder; medical history of the patient, age, body weight, general health, sex and diet of the patient, the time of administration, route of administration, the duration of the treatment, other drugs being taken by the patient, and the like. A single administration may usually be sufficient to produce a therapeutic effect, but it is contemplated that multiple administrations will be used over a substantial period of time to assure a continued sustained response.

Accordingly, the present invention provides a single-directional insufflator or breath-powered nasal device 10 for delivering a drug substance, in powder or liquid form, to the nasal airway of a subject, comprising: a closure unit for causing the closure of the oropharyngeal velum of the subject; and a dual wall drug system 30 that provides dual airflow for delivering a vertical, somewhat tightly and narrow or confined, concentrated plume of air flow entraining a drug substance to one of the nostrils of the subject at a driving pressure which would be breath powered to drive the confined and concentrated plume deep within the nasal cavity for targeting the olfactory region, including the olfactory nerve, and the trigeminal nerve mucosa for direct diffusion into the brain and for effective treatment of nasal and/or CNS injury, disease or disorder, in particular TBI, especially concussion, within minutes of nasal application, wherein a single-directional insufflator or breath-powered nasal device 10 of the present invention comprises a nose guard 20 which includes an outlet through which the breath powered air flow is delivered to the one nostril for providing a fluid tight seal between the nose guard 20 and the one nostril.

More specifically, single-directional insufflator or breath-powered nasal device 10 of the present invention comprises a nose guard 20, a one-way valve mouth piece 100, corrugated tubing 80 and means for propelling a dual airflow, e.g., dual wall drug dispensing system 30, i.e., a double-walled tube configuration, for propelling a first airflow carrying the powdered drug substance and propelling a second airflow for surrounding the first airflow for propelling highly concentrated powdered drug substance deep within the nasal cavity, as shown, for example, in FIGS. 1 and 2.

More specifically speaking, the single-directional insufflator or breath-powered nasal device 10 of the present invention is comprised of nose guard 20, dual wall drug dispensing system 30, i.e., a double-walled tube configuration, mesh disc 40, first tube connector 60, corrugated tubing 80, second tube connector 70, and a one-way valve mouth piece 100, as shown in FIGS. 1-2 and 7-10.

The nose guard 20 is tapered and configured with a through-hole 24 and an internal collar 26 against which the top portion 31 of the dual wall drug dispensing system 30 rest when the dual wall drug dispensing system 30, i.e., a double-walled tube configuration, is fully inserted into aperture 24 of nose guard 20, as shown in FIGS. 9-10 and 23-24. Nose guard 20 can be manufactured out of any material that is suitable for nose guard's 20 intended purposes. Seal cap 150, i.e., top cover, as shown in FIGS. 1, 9-10, 13-14 and 26-28, may be inserted into the top 25 of aperture 24 to seal nose guard 20.

The dual wall drug dispensing system 30, is a novel and unique system for delivering one or more drug substances into the nasal cavity and an important aspect of the present invention. The dual wall drug dispensing system 30, i.e., a double-walled tube configuration, is preferably extruded and comprises a top end portion 31 and the bottom end portion 32 and two hollow chambers 33, an inner hollow chamber 33a and an outer hollow chamber 33b. The hollow inner chamber 33a holds drug substance 200 (not shown). The hollow outer chamber 33b, which surrounds the hollow inner chamber 33a, is divided into two, three, four or more separate chambers 34 by rib walls 35. Preferably, hollow outer chamber 33b is divided into three separate chambers 34. See, for example, FIGS. 30-32.

Bridge connector 110 connects one-way valve mouth piece side 131 comprising the one-way valve mouth piece 100 and second tube connector 70 to nose guard side 130 comprising the nose guard 20, dual wall drug dispensing system 30, i.e., a double-walled tube configuration, mesh disc 40, first tube connector 60 and corrugated tubing 80 to maintain the breath-powered insufflator or breath-powered nasal device 10 of the present invention in a constant “U-shape” configuration 140. Bridge connector 120 comprises a bridge 121 sandwiched between two hollow rings 122 and 123, respectively, for sliding over first hollow cuff 81 and second hollow cuff 82, respectively of convoluted middle section with corrugations 80, as shown in FIGS. 1-4 and 7-12, 16-17-19, 21-22, 24-26 and 28.

Alternatively, the bridge connector 120 may be designed with a perforation or score line 124 or any other configuration that allows bridge connector 120 to be broken in two pieces in the event sides 130 and 131 of the device need to be straightened for use by a second person who is assisting, e.g., a concussed person who cannot self-administer the necessary blow force to provide sufficient airflow to effectively propel the drug substance from the single-directional insufflator or breath-powered nasal device 10 of the present invention to effectively treat the concussion with the drug substance. See FIGS. 13 and 14.

The dual wall drug dispensing system 30, i.e., a double-walled tube configuration, is a novel and unique system for delivering one or more drug substances into the nasal cavity and an important aspect of the present invention. The dual wall drug dispensing system 30, i.e., a double-walled tube configuration, comprises a top end portion 31 and the bottom end portion 32 and two hollow chambers 33, an inner hollow chamber 33a and an outer hollow chamber 33b. The hollow inner chamber 33a holds drug substance 200. The hollow outer chamber 33b, which surrounds the hollow inner chamber 33a, is divided into two, three, four or more separate chambers 34 by rib walls 35. Preferably, hollow outer chamber 33b is divided into three separate chambers 34. See, for example, FIGS. 29-32.

First and second tube connectors 60 and 70 are each provided with first top hollow collar 65 and second top hollow collar 75, respectively, for insertion into first hollow cuff 81 and second hollow cuff 82, respectively, of corrugated tubing 80. However, before first top hollow collar 65 and second top hollow collar 75 are inserted into respective first hollow cuff 81 and second hollow cuff 82 of corrugated tubbing 80, bridge connector 110 the first hollow ring 122 and second hollow ring 123 of bridge connector 110 is slid over first hollow cuff 81 and second hollow cuff 82, respectively, to create a u-shape configuration 140. See FIGS. 16-17.

As described herein before, to load the drug substance 200, when in a powdered format, especially a spray-dried format, into the novel single-directional insufflator or breath-powered nasal device 10 of the present invention, a sterilized funnel (not shown) is used to load the powdered drug substance 200 specifically within the inner hollow chamber 33a of the dual wall drug dispensing system 30, i.e., a double-walled tube configuration. The mesh disc 40, which is positioned at bottom end portion 32 of the inner hollow chamber 33a, at the top 66 of the first tube connector 60, is what the loaded powdered drug substance 200 first encounters and against which it rest once fully loaded. Because the pore size of the mesh disc 40 is smaller than the particle size of the powdered drug substance, the powdered drug substance cannot fall through the mesh disc 40. The pores throughout the mesh disc 40, however, permit the blown airflow to penetrate up and through the mesh disc 40 to carry and propel the powdered drug substance 200 from the inner hollow chamber 33a out of nose guard 20 and into the nasal cavity.

Mesh disc 40, as shown for example in FIG. 1, can be manufactured from a multitude of different material options. For example, mesh disc 40 can be manufactured with a PET material. Different materials and mesh size could be used for different applications. By way of example, it may be possible to use liquid with use of a PTFE hydrophobic membrane to contain the dose (depending on chemistry of course). One example of a material contemplated by the present invention for mesh disc 40 is SEFAR MEDIFAB 07-135/40 158 cm, Item Number: 3A07-0135-158-00. One example of a sized mesh disc 40 manufactured with the SEFAR MEDIFAB 07-135/40 158 cm, Item Number: 3A07-0135-158-00 material, that is contemplated for use in accordance with the present, is a mesh disc 40 having a diameter of about 0.411, a thickness of from about 0.004 inches to about 0.012 inches or more, and a surface roughness of about 125 μin.

Preferably, the particle size distribution of the powdered, spray-dried, drug substance 200 is principally in the range of about 1 μm to about 10 μm. In one embodiment, the powdered, spray-dried, drug substance contains a medicament, particularly for the treatment of a nasal condition. Notwithstanding the preferred particle size, the present invention contemplates particle size distribution of a powdered, spray-dried, drug substance to include larger particles, or a smaller fraction of larger particles, typically in the range of about 10 μm to about 30 μm, and preferably in the range of about 20 μm to about 30 μm.

By nesting the inner hollow chamber 33a with powdered, spray-dried, drug substance 200 (not shown) inside the unobstructed larger outer hollow chamber 33b, as shown FIGS. 1-2, 7- and 30, dual wall drug dispensing system 30, i.e., a double walled-tubing configuration, will allow for unmedicated airflow to engage the sidewall of the nasal cavity while allowing the airflow carrying the powdered, spray-dried, drug substance 200, which flows in the center of the unmedicated airflow, to reach its targeted destination deep inside the nasal cavity with minimal drug substance 200 loss.

This benefit is illustrated in FIGS. 48-51, where there is little dry powder build up on the inner walls 332 of inner hollow chambers 33a of dual wall drug dispensing systems 30, i.e., a double walled-tubing configuration, post-powder spray through inner hollow chambers 33a, as compared to the significant dry powder build up on the inner walls of single tube constructions with 3 mm or 4 mm baseline, FIG. 46, and the significant dry powder build up on the inner wall of single tube construction with mm baseline, FIG. 47, and making both tubes almost opaque, post-powder spray through the single tube construction. In contrast, and as shown in FIGS. 48-51, the inner walls 332 of inner hollow chambers 33a of dual wall drug dispensing systems 30, i.e., a double walled-tubing configuration, with 1 mm gap, 1.5 mm gap, 2 mm gap and 3 mm gap, respectively, have a light dusting, with very little powder build up, post-powder spray through inner hollow chamber 33a indicating that more dry powder reaches the targeted destination.

One-way valve mouth piece 100, as shown in FIGS. 1-2, 9-10 and 28, comprises there through conduit 101 with top section 102 and bottom section 103, respectively. Within bottom section 103 sits a one-way valve (not shown) for directing blown air into corrugated tube 80 and preventing reverse airflow. One-way valve mouth piece 100 is designed for insertion into second collar 75 of second tube connector 70 against which bottom section 103 sits when one-way valve mouth piece 100 is fully press-fit into second collar 75 of second tube connector 70. An example of a one-way valve mouth piece 100 contemplated by the present invention is Crosstex Safe-Flo™ Adapter, M1020ADAP one-way valve mouth piece manufactured by Crosstex International. The Safe-Flo™ Adapter is designed with a unique, built-in one-way valve to provide a physical barrier that prevents backflow.

Corrugated tubing 80 is arched to a degree that allows nose guard side 130 and 131 to form a U-shaped configuration 140, as shown in FIGS. 1-2, 7-8, 13, 11, 13 and 37. Corrugated middle section 85 of corrugated tubing 80 has first and second top ends 811 and 821 for respectively receiving first bottom hollow neck 66 of first tube connector 60 and second bottom hollow neck 76 of second tube connector 70 to further the assembly of the single-directional insufflator or breath-powered nasal device 10 of the present invention, as shown in FIGS. 15 and 18.

FIG. 1 is a front assembled elevational angled front view 135 of a single-directional insufflator or breath-powered nasal device 10.

FIG. 2 is a rear assembled elevational angled back view 136 of a single-directional insufflator or breath-powered nasal device 10.

FIG. 3 is a top view 850 of a corrugated tube 80 showing corrugated middle section 85 and bridge connector 110.

FIG. 4 is a bottom view 860 of a corrugated tube 80 showing corrugated middle section 85.

FIG. 5 is an assembled elevational view of nose guard side 130 of the single-directional insufflator or breath-powered nasal device 10.

FIG. 6 is an assembled elevational view of the one-way valve mouth piece side 131 of the single-directional insufflator or breath-powered nasal device 10.

FIG. 7 is a right mouth piece assembled elevational view of a single-directional insufflator or breath-powered nasal device 10.

FIG. 8 is a left mouth piece assembled elevational view of a single-directional insufflator or breath-powered nasal device 10.

FIG. 9 is a right mouth piece front unassembled view of a single-directional insufflator or breath-powered nasal device 10.

FIG. 10 is a left mouth piece front unassembled view of a single-directional insufflator or breath-powered nasal device 10.

FIG. 11 illustrates single-directional insufflator or breath-powered nasal device 10, as shown in FIGS. 1-2 and 4-8, inserted into a nasal cavity of a subject for operation.

FIG. 12 illustrates nose guard 20 of a single-directional insufflator or breath-powered nasal device 10, as shown in FIGS. 13 and 14, that is inserted into a nasal cavity of a first subject to be treated with a drug substance 200 (not shown) and one-way valve mouth piece 100, as shown in FIGS. 13 and 14, inserted between the lips of a second subject for operation of the alternative embodiment by the second subject as shown in FIGS. 13 and 14.

FIG. 13 is a front, right mouth piece, assembled elevational angled view of a single-directional insufflator or breath-powered nasal device 10, wherein the bridge connector 110 is designed with a perforated or score line 142 for breaking bridge connector 110 in two.

FIG. 14 is a front, right mouth piece, assembled elevational angled view of a single-directional insufflator or breath-powered nasal device 10, wherein the bridge connector 110 is broken in two pieces at the perforated or score line 142 for breaking bridge connector 110 into two separate pieces, as shown in FIG. 13.

FIG. 15 illustrates assembling bridge connector 110 to corrugated tubing 80.

FIG. 16 illustrates a perspective view of bridge connector 110 and corrugated tubing 80 in an assembled configuration.

FIG. 17 illustrates a front view of bridge connector 110 and corrugated tubing 80 in an assembled configuration.

FIG. 18 illustrates assembling first tube connector 60 and second tube connector 70 to corrugated tubing 80 assembled with bridge connector 110, as shown in FIGS. 16 and 17.

FIG. 19 illustrates a perspective view of first tube connector 60 and second tube connector 70 assembled with corrugated tubing 80 assembled with bridge connector 110, as shown in FIGS. 16 and 17.

FIG. 20 illustrates a cross-sectional view of tube connectors 60 and 70.

FIG. 21 illustrates inserting a mesh disc 40 into first tube connector 60, wherein first tube connector 60 and second tube connector 70 are assembled with corrugated tubing 80 assembled with bridge connector 110, as shown in FIGS. 16 and 17.

FIG. 22 illustrates inserting a dual wall drug dispensing system 30, i.e., a double-walled tube configuration, into first tube connector 60, wherein mesh disc 40 is inserted into first tube connector 60 and first tube connector 60 and second tube connector 70 are assembled with corrugated tubing 80 assembled with bridge connector 110, as shown in FIGS. 16, 17, 19 and 21.

FIG. 23 illustrates an exploded cross-sectional view of assembled first tube connector 60, wherein mesh disc 40 and dual wall drug dispensing system 30, i.e., a double-walled tube configuration, are assembled within first tube connector 60 and mesh disc 40 is sitting on first seat 61 of first tube connector 60 and first top hollow collar 65 of first tube connector 60 is press-fit into first hollow cuff 81 of corrugated tubing 80.

FIG. 24 illustrates inserting top end portion 31 of dual wall drug dispensing system 30, i.e., the double walled-tube configuration, into bottom 26 of through-hole 24 of nose guard 20, wherein mesh disc 40 is assembled within first tube connector 60 and first tube connector 60 and second tube connector 70 are assembled with corrugated tubing 80 assembled with bridge connector 110, as shown in FIGS. 16, 17, 19, 21 and 22.

FIG. 25 illustrates a front view of assembled nose guard 20, first tube connector 60, mesh disc 40 (not shown), dual wall drug dispensing system 30, i.e., a double-walled tube configuration, first tube connector 60 and second tube connector 70, bride connector 110 and corrugated tubing 80.

FIG. 26 illustrates placing seal cap 150 with seal cap plug 151 into top 25 (not shown) of through-hole 24 (not shown) over nose guard 20 of assembled nose guard 20, first tube connector 60, mesh disc 40 (not shown), dual wall drug dispensing system 30, i.e., a double-walled tube configuration, first tube connector 60 and second tube connector 70, bride connector 110 and corrugated tubing 80.

FIG. 27 illustrates an exploded cross-sectional view of nose guard 20, wherein dual wall drug dispensing system 30, i.e., the double walled-tube, is inserted through through-hole 24 of nose guard 20, top end portion 31 of dual wall drug dispensing system 30, i.e., a double-walled tube configuration, is seated against nose guard seat 22 of nose guard 20 and seal cap 150 is positioned over nose guard 20 with nose guard stem 151 is inserted through top 25 and into through-hole 24 of nose guard 20.

FIG. 28 illustrates inserting bottom section 102 of one-way valve mouth piece 100 into of dual wall drug dispensing system 30, i.e., the double walled-tube, into second top hollow collar 75 of second tube connector 70, assembled seal cap 150, nose guard 20, first tube connector 60, mesh disc 40 (not shown), dual wall drug dispensing system 30, i.e., a double-walled tube configuration, first tube connector 60 and second tube connector 70, bride connector 110 and corrugated tubing 80.

FIG. 29 is an exploded cross-sectional view of inner hollow chamber 33a, having outer wall 331 and inner wall 332, and outer hollow chamber 33b having outer wall 333 and inner wall 334 of dual wall drug dispensing system 30, i.e., a double-walled tube configuration, illustrating one size tube diameters, wherein, outer hollow chamber 33b having outer wall 333 diameter of 11 mm and inner wall 334 diameter of 9 mm and inner hollow chamber 33a having outer wall 331 diameter of 6 mm and inner wall 332 diameter of 4 mm with internal gap 36 of 1.5 mm and an inner hollow chamber 33a having an internal diameter of 4 mm. Illustrations of alternative size tube diameters are: (a) outer hollow chamber 33b having outer wall 333 diameter of 10 mm and inner wall 334 diameter of 8 mm and inner hollow chamber 33a having outer wall 331 diameter of 6 mm and inner wall 332 diameter of 4 mm with internal gap 36 of 1 mm and an inner hollow chamber 33a having an internal diameter of 4 mm; (b) outer hollow chamber 33b having outer wall 333 diameter of 11 mm and inner wall 334 diameter of 9 mm and inner hollow chamber 33a having outer wall 331 diameter of 5 mm and inner wall 332 diameter of 3 mm with internal gap 36 of 2 mm and an inner hollow chamber 33a having an internal diameter of 3 mm; and (c) outer hollow chamber 33b having outer wall 333 diameter of 13 mm and inner wall 334 diameter of 11 mm and inner hollow chamber 33a having outer wall 331 diameter of 5 mm and inner wall 332 diameter of 3 mm with an internal gap 36 of 3 mm and an inner hollow chamber 33a having an internal diameter of 3 mm.

FIG. 30 is a view of dual wall drug dispensing system 30, i.e., a double-walled tube configuration, bottom end portion 32, internal gap 36 and the two hollow chambers 33, i.e., the inner hollow chamber 33a and the outer hollow chamber 33b, wherein the outer hollow chamber 33b is divided into three separate chambers 34 by rib walls 35.

FIG. 31 is a cross-sectional view of dual wall drug dispensing system 30, i.e., a double-walled tube configuration, as shown in FIGS. 9, 10, 19 and 30.

FIG. 32 is a cross-sectional view taken along line A of dual wall drug dispensing system 30, i.e., a double-walled tube configuration, as shown in FIGS. 9, 10, 19, 30 and 31.

FIG. 33 is a cross-sectional view of tube connector 60 or 70 taken along line A, as shown in FIGS. 9, 10, 18, 23 and 31.

FIG. 34 is a cross-sectional view of tube connector 60 or 70, as shown in FIGS. 9, 10, 18, 23 and 31.

FIG. 35 is a cross-sectional view of nose guard 20 taken along line A depicting top 25 of through-hole 24, as shown in FIGS. 9, 10, 24, 25 and 27.

FIG. 36 illustrates an alternative cross-sectional view of nose guard 20, depicting top 25 and bottom 26 of through-hole 24 and nose guard seat 22, as shown in FIGS. 9, 10, 24, 25, 27 and 35.

FIG. 37 illustrates an assembled view of single-directional insufflator or breath-powered nasal device 10 depicting one-way valve mouth piece 100, first air-flow directional band 160, first tube connector 60, corrugated tubing 80, bridge connector 110, second tube connector 70, second air-flow directional band 170, dual wall drug dispensing system 30 and nose guard 20.

FIG. 36 illustrates an alternative one-way valve mouth piece 131 view of and assembled single-directional insufflator or breath-powered nasal device 10 nose guard, as shown in FIG. 6.

FIG. 39 illustrates an unassembled view of seal cap 150, i.e., top plug, nose guard 20, one-way valve dual wall drug dispensing system 30, i.e., a double-walled tube configuration, mesh disc 40, and alternative connector 180 for connecting one-way dual wall drug dispensing system 30, i.e., the double walled-tube, and corrugated tubing 80.

FIG. 40 illustrates an alternative cross-sectional view of seal cap 150, i.e., top plug, nose guard 20, and one-way valve dual wall drug dispensing system 30, i.e., a double-walled tube configuration, assembled with alternative connector 180, as shown in FIG. 39;

FIG. 41 illustrates an assembled front view of nose guard 20, dual wall drug dispensing system 30, i.e., a double-walled tube configuration, alternative connector 180, corrugated tubing 80, and nose guard 20.

FIG. 42 illustrates a top view of a tray packaged with twenty single-directional insufflator or breath-powered nasal fully assembled devices herein, wherein the 20 such devices 10 are positioned in two rows of five pairs of such devices, wherein each such pair shows two such devices positioned in an interlocking position with respect to one another.

FIG. 43 illustrates a side top view of a tray packaged exposing an internal side longitudinal wall with twenty single-directional insufflator or breath-powered nasal fully assembled devices herein, wherein the 20 such devices 10 are positioned in two rows of five pairs of such devices, wherein each such pair shows two such devices positioned in an interlocking position with respect to one another.

FIG. 44 illustrates a screen shot comparing the width of a lactose powder spray between a single tube configuration and a dual wall drug dispensing system 30, i.e., a double-walled tube configuration, of the present invention in which the internal diameter of the inner tube of the double tube is about 4 mm and the gap between the inner and outer tubes of the double tube is either 1.5 mm or 1 mm to a single tube configuration.

FIG. 45 illustrates a screen shot comparing the width of a lactose powder spray between a single tube configuration and a dual wall drug dispensing system 30, i.e., a double-walled tube configuration, of the present invention in which the internal diameter of the inner tube of the double tube is about 3 mm and the gap between the inner and outer tubes of the double tube is either 2 mm or 3 mm to a single tube configuration.

As shown in FIGS. 44 and 45, when comparing the width of the powder spray at the same height, the spray width of dual wall drug dispensing system 30, i.e., a double-walled tube configuration, with internal diameters of 4 mm and 3 mm, respectively, of inner hollow chambers 33a stays more centered that that of a baseline single tubular configuration. In other words, the baseline single tubular configuration spread out wider and sooner than dual wall drug dispensing system 30, i.e., a double-walled tube configuration. These results demonstrate that dual wall drug dispensing system 30, i.e., a double-walled tube configuration, would allow drug substance 200 (not shown) to possibly reach deeper into a nasal cavity, rather than collecting on the walls of the nose. In addition, because it is believed that the 3 mm ID devices 30 may disperse spray powder at lower heights and reach lower maximum heights than the 4 mm ID devices 30 of FIGS. 44-45, dual wall drug dispensing system 30, i.e., a double-walled tube configuration of the present invention that are configured with inner hollow chambers 33a that have internal diameters of about 4 mm ID or greater are preferred.

FIG. 46 illustrates a screen shot of a single tube configuration with a 3 mm baseline following a lactose powder spray showing significant build up or accumulation of lactose powder on internal wall post-powder spray, as compared to the single tube configuration as shown in FIG. 45.

FIG. 47 illustrates a screen shot of a single tube configuration with a 4 mm baseline following a lactose powder spray showing significant build up or accumulation of lactose powder on internal wall post-powder spray, as compared to the single tube configuration as shown in FIG. 45 . . .

FIG. 48 illustrates a screen shot of a dual wall drug dispensing system 30, i.e., a double-walled tube configuration, with a 1 mm gap following a lactose powder spray showing little build up or accumulation of lactose powder on inner wall 332 of inner hollow chamber 33a post-powder spray, as compared to the single tube configuration as shown in FIG. 45.

FIG. 49 illustrates a screen shot of a dual wall drug dispensing system 30, i.e., a double-walled tube configuration, with a 1.5 mm gap following a lactose powder spray showing little build up or accumulation of lactose powder on inner wall 332 of inner hollow chamber 33a post-powder spray, as compared to the single tube configuration as shown in FIG. 45.

FIG. 50 illustrates a screen shot of a dual wall drug dispensing system 30, i.e., a double-walled tube configuration, with a 2 mm gap following a lactose powder spray showing little build up or accumulation of lactose powder on inner wall 332 of inner hollow chamber 33a post-powder spray, as compared to the single tube configuration as shown in FIG. 45.

FIG. 51 illustrates a screen shot of a dual wall drug dispensing system 30, i.e., a double-walled tube configuration, with a 3 mm gap following a lactose powder spray showing little build up or accumulation of lactose powder on inner wall 332 of inner hollow chamber 33a post-powder spray, as compared to the single tube configuration as shown in FIG. 45.

It should be understood that each part connection described herein is an airtight connection to ensure that blown airflow does not escape and travels in a harmonius single direction.

It should be further understood that top 25 of through-hole 24 of nose guard 20 and top 102 of aperture 101 of one-way valve mouth piece 100 may each be sealed with any effective sealing means to seal nose guard 20 and one-way valve mouth piece 100 from external air or other contamination and moisture, such as a top plug 150 designed to seal top 25 of through-hole 24 of nose guard 20 and top section 102 of conduit 101 when sized to fit accordingly. The present invention also contemplates other suitable sealing means that can serve as alternatives to top plug 150, such as seals using induction foil or shrink wrap, so long as the objectives of the present invention are accomplished and not defeated.

In a preferred embodiment, where the substance is in solid form, such as a powder, the present invention contemplates use of a filter or desiccant if high humidity represents a problem for administration of the solid.

The drug substance 200 can be a single compound or a mixture of compounds, which compounds can be in any suitable form, such as a powder form, a solution, a suspension, a gel or the like.

The drug substance 200 can be any suitable substance for delivery to a human or in some cases an animal. The drug substance 200 may be for delivery for action deep within the nasal cavity for direction diffusion into the brain and/or for topical or systemic action that may require nasal administration of a drug substance 200 for treatment. Thus, a drug substance 200 may be for systemic delivery for action in a region remote from the nasal airway.

Preferably, the drug substance 200 is for delivery to the olfactory region and the trigeminal nerve mucosa for rapid diffusion into the brain for the treatment of nasal and/or CNS injury, disease or disorder, especially brain injury, such as TBI (mild, moderate and/or severe), including concussion.

The drug substance 200 may have a beneficial medical effect, which can include a diagnostic effect, a therapeutic effect, a curative effect, a systemic effect and/or a prophylactic effect, etc. Preferably, the drug substance 200 has a therapeutic or curative effect.

Preferably, the drug substance 200 is a pharmaceutical. The pharmaceutical can be admixed with any suitable carrier, diluent, excipient or adjuvant to form a pharmaceutical composition.

There are many nasal conditions which require treatment. One such condition is brain trauma known, i.e., mild, moderate and severe TBI, including concussion. Another such condition is nasal inflammation, specifically rhinitis, which can be allergic or non-allergic and is often associated with infection and prevents normal nasal function. By way of example, allergic and non-allergic inflammation of the nasal airway can typically effect between 10 and 20% of the population, with nasal congestion of the erectile tissues of the nasal concha, lacrimation, secretion of watery mucus, sneezing and itching being the most common symptoms. As will be understood, nasal congestion impedes nasal breathing and promotes oral breathing, leading to snoring and sleep disturbance. Worryingly, the incidence of such allergic and non-allergic inflammatory diseases is increasing. Other nasal conditions include nasal polyps which arise from the paranasal sinuses, hypertrophic adenoids, secretory otitis media, sinus disease and reduced olfaction.

In the treatment of certain nasal conditions, the topical administration of medicaments is preferable, particularly where the nasal mucosa, especially the olfactory region and distribution into the mucosa innervated by the trigeminal nerve in high drug substance concentration for rapid diffusion into the brain is the prime pathological pathway, such as in treating or relieving CNS injury, disease or disorders. Indeed, topical administration is advantageous in minimizing the possible side effects of systemic administration. Pharmaceutical compositions that are commonly topically delivered include decongestants, antihistamines, cromoglycates, steroids, including C-20 steroids, and antibiotics.

There are now an increasing number of adults and children who rely on pharmaceuticals to relieve symptoms associated with nasal conditions. At present, among the known anti-inflammatory pharmaceuticals, topical steroids have been shown to have an effect on nasal congestion. Topical decongestants have also been suggested for use in relieving nasal congestion. The treatment of hypertrophic adenoids and chronic secretory otitis media using topical decongestants, steroids and anti-microbial agents, although somewhat controversial, has also been proposed. Further, the topical administration of pharmaceuticals has been used to treat or at least relieve symptoms of inflammation in the anterior region of the nasopharynx, the paranasal sinuses and the auditory tubes.

Furthermore, medicaments are now increasingly systemically delivered through the nasal pathway, the nasal pathway offering a good administration route for the topical, systemic and CNS delivery of pharmaceuticals, such as steroids, including C-20 steroids, hormones, for example oxytocin, and anti-migraine compositions, as the high blood flow and large surface area of the nasal mucosa advantageously provides for rapid systemic uptake.

Preferably, the pharmaceutical composition is for the treatment of any one or more of the above-mentioned conditions. By way of example, the pharmaceutical composition may be for the treatment of any allergic and non-allergic inflammatory disease.

Typical pharmaceutical compositions for nasal administration in accordance with the present invention include, but are not limited to, steroids, e.g., C-20 steroids, anti-histamines, cromoglycates, anti-allergic pharmaceuticals, anti-inflammatory pharmaceuticals, anti-leukotrienes, lactation promoters, such as oxytocin, and anti-migraine pharmaceuticals.

By achieving a more optimal and, optionally broad drug substance spectrum, nasal delivery, the nasal insufflator art through the discovery of a novel single-directional insufflator or breath-powered nasal device and methods improves the effect of topical pharmaceuticals in the preferable treatment of CNS injury, disease or disorder, especially TBI, and most preferably concussion.

It should be understood that any reference, document, patent, patent publication or the like cited or disclosed herein, including articles and websites, are incorporated herein by reference in their entireties as if fully set forth herein.

The present disclosure has been described with reference to example embodiments. Modifications and alterations will occur to others of skill in the art upon reading and understanding the preceding detailed description. It is intended that the present disclosure be construed as including all such modifications and alterations insofar as they come within the scope of the appended claims or the equivalents thereof.

### DESCRIPTION OF NUMERICAL LEGENDS

single-directional insufflator or breath-powered nasal device 10
nose guard 20
   nose guard seat 22
   through-hole 24
      top 25
      bottom 26
dual wall drug dispensing system 30, i.e, a double walled-tubing configuration
   top end portion 31
   bottom end portion 32
   two hollow chambers 33
      inner hollow chamber 33a
         outer wall 331
         inner wall 332
      outer hollow chamber 33b
         outer wall 333
         inner wall 334
   separate chambers 34
   rib walls 35
   internal gap 36
mesh disc 40
one-way valve 50 (not shown)
first tube connector 60
   first seat 61
   first top hollow collar 65
   first bottom hollow neck 66
second tube connector 70
   second seat 71
   second top hollow collar 75
   second bottom hollow neck 76
corrugated tubing 80
   first hollow cuff 81
      first top end 811
   second hollow cuff 82
      Seconds top end 821
   corrugated middle section 85
   top view 850
   bottom view 860
one-way valve mouth piece 100
   conduit 101
   top section 102
   bottom section 103
bridge connector 110
   bridge 121
   first hollow ring 122
   second hollow ring 123
   perforation or score line 124
nose guard side 130
one-way valve mouth piece side 131
front view of single-directional insufflator or breath-powered nasal device 135
back view of single-directional insufflator or breath-powered nasal device 136
drug substance 200 (not shown)
U-shaped configuration 140
seal cap 150, i.e., top cover
   seal cap plug 151
first air-flow directional indicator band 160
second air-flow directional indicator band 170
alternative connector 180

Having described our invention, we claim:

1) A novel single-directional breath-powered nasal device for providing dual airflow for propelling a drug substance, when the drug substance is loaded into said device, into a nasal cavity for targeted delivery to the olfactory region in high drug substance concentration for rapid diffusion into the brain for the treatment of local or systemic nasal and/or central nervous system (“CNS”) injury, disease or disorder, said device comprises:
(a) a mouse piece for blowing a breath into said device by a subject;
(b) a nose guard for propelling the drug substance in the nasal cavity of the subject;
(c) a flexible convoluted middle section sandwiched between said mouth piece and nose guard; and
(d) a dual wall drug dispensing system comprised of a first hollow means into which the drug substance is loaded that generates a first airflow, and a second hollow means that surrounds the first hollow means, so that, when a subject blows air through said mouthpiece and into said device, said first hollow means and said second hollow means in parallel provide a dual airflow comprised of (i) a first airflow expelled from said first hollow means carrying the drug substance, and (ii) a second airflow expelled from the second hollow means to create airflow pressure that surrounds the first expelled airflow, so that the first expelled airflow forms and travels initially in a vertical, somewhat tight and narrow or confined, concentrated plume that drives the concentrated drug substance to the targeted olfactory region for direct diffusion into the brain for the effective treatment.

2) A device of claim 1, wherein the drug substance is deposited into the superior nasal cavity.

3) A device of claim 1, wherein the drug substance is distributed into mucosa innervated by the trigeminal nerve.

4) A device of claim 1, wherein the CNS injury is brain injury.

5) A device of claim 1, wherein the brain injury is traumatic brain injury (“TBI”).

6) A device of claim 1, wherein the TBI is mild TBI, moderate TBI or severe TBI.

7) A device of claim 4, wherein the brain injury is a concussion.

8) A device according to any one of claim 1, wherein the drug substance is ent-19-norprogesterone.

9) A device according to any one of claim 2, wherein the drug substance is ent-19-norprogesterone.

10) A device according to any one of claim 3, wherein the drug substance is ent-19-norprogesterone.

11) A device according to any one of claim 4, wherein the drug substance is ent-19-norprogesterone.

12) A device according to any one of claim 5, wherein the drug substance is ent-19-norprogesterone.

13) A device according to any one of claim 6, wherein the drug substance is ent-19-norprogesterone.

14) A device according to any one of claim 7, wherein the drug substance is ent-19-norprogesterone.

15) A method of treating a subject for local or systemic nasal injury, disorder or disease or CNS injury, disorder or disease, who is in need of treatment, said method comprises:
(a) using the device of claim 1 to deliver a therapeutically effective concentrated amount of drug substance to the olfactory region of a subject for rapid diffusion into the brain for the treatment of the local or systemic nasal injury, disorder or disease and/or CNS injury, disease or disorder.

16) A method of claim 9, wherein the concentrated drug substance is deposited into the superior nasal cavity.

17) A method of claim 9, wherein the concentrated drug substance is distributed into mucosa innervated by the trigeminal nerve.

18) A method of claim 9, wherein the CNS injury is brain injury.

19) A method of claim 9, wherein the brain injury is traumatic brain injury (“TBI”).

20) A method of claim 9, wherein the TBI is mild TBI, moderate TBI or severe TBI.

21) A method of claim 12, wherein the brain injury is a concussion.

22) A method according to claim 9, wherein the drug substance is ent-19-norprogesterone.

23) A method according to claim 10, wherein the drug substance is ent-19-norprogesterone.

24) A method according to claim 11, wherein the drug substance is ent-19-norprogesterone.

25) A method according to claim 12, wherein the drug substance is ent-19-norprogesterone.

26) A method according to claim 13, wherein the drug substance is ent-19-norprogesterone.

27) A method according to claim 12, wherein the drug substance is ent-19-norprogesterone.

28) A method according to claim 13, wherein the drug substance is ent-19-norprogesterone.

29) A method according to claim 14, wherein the drug substance is ent-19-norprogesterone.

30) A method according to claim 15, wherein the drug substance is ent-19-norprogesterone.

31) A method according to claim 16, wherein the drug substance is ent-19-norprogesterone.

32) A method according to claim 17, wherein the drug substance is ent-19-norprogesterone.

33) A method according to claim 18, wherein the drug substance is ent-19-norprogesterone.

34) A method according to claim 19, wherein the drug substance is ent-19-norprogesterone.

35) A method according to claim 20, wherein the drug substance is ent-19-norprogesterone.

36) A method according to claim 21, wherein the drug substance is ent-19-norprogesterone.
